(19) United States
(12) Patent Application Publication
(10) Pub. No.: US 2020/0110736 A1
(43) Pub. Date: Apr. 9, 2020

(54) Natural language, flat field, record management and file system that defines, integrates and operates records comprising best practices and establishes collaborative peer networks to evolve new best practice records

(71) Applicant: Robert Paul Bauman, Cleveland, OH (US)

(72) Inventor: Robert Paul Bauman, Cleveland, OH (US)

(21) Appl. No.: 16/501,328

(22) Filed: Mar. 28, 2019

Related U.S. Application Data
(60) Provisional application No. 62/649,883, filed on Mar. 29, 2018.

(51) Int. Cl. G06F 16/22; G16H 10/60

(52) U.S. Cl. CPC G06F 16/2246; G16H 10/60

(57) ABSTRACT

A record system provides improved capabilities in healthcare organizations and other enterprises. The record system provides a natural language, flat field record management and file system that defines, integrates and operates using records that reflect best practices. The system further operates to establish collaborative peer networks and to evolve records which reflect best practices of the organization.

### TECHNICAL FIELD

Exemplary embodiments relate to a record storage system. Specifically exemplary embodiments relate to a record storage system that may be used in healthcare organization environments as well as other environments to provide enhanced capabilities and improve system operations.

### BACKGROUND

Large organizations typically have various types of information technology systems that are utilized in the conduct of their activities. Often the systems include different architectures and platforms which make it difficult to access all of the necessary data and capabilities that may be available from the different systems.

Such systems are often designed with structured functionality that cannot be readily changed to suit the needs of particular individual users or adapted to address new requirements which may arise. In addition it is often difficult to modify such systems to suit the needs of a local site or a particular operational function within the organization which may need additional or different systems capabilities. Systems can also be complex and difficult to update or improve without the need to make extensive revisions using professional programmers.

The above described drawbacks are often particularly significant in the healthcare field. It is common to find that numerous different systems are operated to track and analyze different types of information that may be related to the care of particular patients.

Such prior systems that are used in the healthcare field and within other organizations may benefit from improvements.

### SUMMARY OF EXEMPLARY EMBODIMENTS

An exemplary embodiment establishes a Record Management System (RMS) that utilizes three delimiter-based storage algorithms that collectively construct a flat field relational database. The database relates delimiter constructed files of attribute-specific elements by means of disposed elements of structure independent of the disposed elements. A respective attribute further includes means to dispose all data comprising an enterprise data system as plain text within a single storage-field or equivalent.

The exemplary RMS establishes three sets of lexicographic, hierarchal delimiters. The delimiters establish three interactive, delimiter-mediated abstract storage constructs that structure the plain-text database to enable RMS functions. The first set of delimiters establishes a record-file respective of each attribute of data and each attribute of function with means to index, label and manage all respective elements comprising a record-file. The second set of delimiters establishes a record-set of record-files with means to index, label and group-store respective related files. The third set of delimiters establishes a matrix construct of delimiters that enables disposing each attribute-specific record-file and each record-set of record-files bracketed within matrix delimiters enabling unlimited matrix-based groupings (W) with each matrix group comprised of unlimited (Z) panels (Tables). The tables are comprised of unlimited Y lines initiating an unlimited X column array of XX bracketed record files and record-sets. The abstract matrix storage design enables uniquely identifying each storage site, and thereby each disposed record-file or record-set of record-files by means of disposing within each respective record-file the respective unique four-component, natural number, (W) and matrix-specific Z-Y-X structural intercept i.e., (W.Z.Y.X).

RMS Natural Language Programming tools (NLP tools) identify and label the requisite elements of RMS storage. The NLP tools activate respective RMS database storage programs that integrate the interdependent delimiter sets collectively enabling precise structuring, indexing and labeling of disposed elements, matrix structured storage and accurate W.Z.Y.X assignments respective of connecting data and related data-driven record-files disparately disposed within a single storage-field or equivalent.

RMS storage design templates ontologically define all data respective of a related data-dependent function, eliminating the ambiguities of plain text and enables the accurate W.Z.Y.X relational assignments.

RMS bottom-up programming templates enable matching the top-down, expert designed, function-supporting data tables of Legacy data systems with record-file interfaces. This enables two-way data exchanges between legacy and RMS data systems and routing common data across RMS connected Legacy data systems eliminating redundant Legacy entries.

The RMS NLP tools empower subject-matter experts who do not have expert programming skills to transform their knowledge-driven data-dependent Legacy functions into automated RMS data-driven functions. This is done by morphing a function-defining, delimiter-based abstract storage structure into a portable, function-specific, operational platform.

The RMS operational platform warehouses function-specific data and cloud-based, generic program identifiers as well as the capability to recruit cloud-based programs to process. analyze and define the site-specific and function-specific data, based on activating a single programmed action or a set of graded programmed actions that complete a defined site-specific function. Data parameters and pre-programmed graded actions are both subject matter expert defined. This establishes a knowledge-warehouse respective of each expert's knowledge and bias respective of best practice.

The exemplary RMS operational platform manages a single function with limited data that enables warehousing expert-specific informative text-data templates respective of a plurality of data-specific parameters. Site-specific data parameters are matched to the appropriate text-data template means for automating the generation of expert-specific, data-specific and site-specific informative text routed to respective Legacy data systems enabling customized efficient documentation.

The RMS enables automated outcome monitoring, acquisition and storage within the respective operational platform, thereby completing site-specific records of data, data parameters, knowledge-directed actions and respective outcomes. These site-specific records fuel automated machine-learning algorithms to optimize site-specific actions and enable self-directed best practice analytics. Aggregate records across all RMS knowledge directed operational functions of the organization enable aggregate enterprise-directed best practice analytics.

The RMS establishes a uniquely disposed Data-Switch (DS) which uses W.Z.Y.X identifiers. The identifiers provide either unique or related means to tag a respective record-file as active to transport or receive data.

The RMS further establishes multitudes of cloud-based, generic, Avatar-connect programs each activated by means of an RMS mediated, W.Z.Y.X assignment typified by new RMS data. RMS activated Avatar-connect programs connect record-files with matched DS disposed W.Z.Y.X identifiers, exemplified by a new data record-file disposing its data-specific W.Z.Y.X identifier within its DS, and a respective function-specific record-file activated to receive the new data by means of redisposing a respective related data-specific W.Z.Y.X identifier into its respective DS. An Avatar-connect between matched DS disposed W.Z.Y.X identifiers, activates the programmed assignments of each respective record-file that enable transferring the new data to a disparate disposed, function-specific, Avatar-connected, record-file.

The exemplary RMS establishes Virtual Assistants for each subject-matter expert and their knowledge-directed programmed actions by automating data-driven functions. Legacy data interfaces enable the RMS to dispose new data within a respective W.Z.Y.X identified record-file and disposes the data-specific W.Z.Y.X identifier into the next available cloud-based Avatar-connect program. This activates the respective Avatar-connection process that sequentially locates each DS-activated function-specific record-file and transfers the respective data that initiates the respective automated data-driven functions. Record development enables expert guidance and governance post activation.

### BRIEF DESCRIPTION OF DRAWINGS

FIG. 1 shows schematically the primary components of a record system of an exemplary embodiment.

FIG. 2 is a schematic representation of features an exemplary record management system.

FIG. 3 is a schematic representation of storage structures used in an exemplary system.

FIG. 4 is a schematic representation of delimiter constructs used for exemplary storage structures.

FIG. 5 schematically represents elements of an exemplary storage record.

FIG. 6 schematically represents the elements of storage records that are expanded to include function elements.

FIG. 7 schematically represents how the exemplary system disposes, locates and moves storage records.

FIG. 8 schematically shows the capabilities of the natural language programming (NLP) tools of an exemplary embodiment.

FIG. 9 schematically represents the operation of the exemplary system in providing configurable visual outputs.

FIG. 10 schematically represents the operation of the exemplary system in generating visual outputs that correspond to outputs produced by legacy systems.

FIG. 11 schematically represents platform chapter components that are used in an exemplary system.

FIG. 12 schematically represents function specific operation platform components.

FIG. 13 schematically represents aspects of an exemplary system that connect data records and function records.

FIG. 14 schematically represents a condensed matrix construct used in an exemplary system.

FIG. 15 schematically represents the matrix storage structure used in an exemplary system.

FIG. 16 schematically represents linear storage delimiter constructs used in an exemplary system.

FIG. 17 schematically represents the extendable matrix construct used with exemplary records.

FIG. 18 schematically illustrates the operation of the exemplary system in building matrix storage.

FIG. 19 schematically illustrates the topography and logic flow of the exemplary system.

### DETAILED DESCRIPTION

The exemplary system embodiments establish a transformational record management system (RMS) that establishes a record-centric, flat-field, linear-storage, alphanumeric-based, digital storage system. This system is comprised of three primary components. Each of the components includes circuits that carry out circuit executable instructions. The first component establishes methods to uniquely code each record with a coding system that encodes storage-architecture means to relate disposed records respective of storage-architecture. The second component establishes methods to code record-specific relationships within a respective record that enable functional relationships between records and between peer groups of records. The third component establishes methods to code record selections with respect to knowledge-based selections. This enables pattern analyses that fuel knowledge-based programming of automated record selection functions to transform knowledge code to automated data-driven functions.

The exemplary system establishes a record respective of each attribute of data or function. These records include the attribute glossaries of each Legacy data system. The exemplary system further includes means to dispose each attribute-specific record separately and together within a single storage-field, or equivalent records regarding each attribute comprising the collective of data, processes and functions of the enterprise in which the system is used. Non-alphanumeric-based elements are titled and referenced for purposes of retrieving from storage that is not part of the exemplary RMS.

The exemplary system further includes capabilities to categorically group, sub group, and sub-sub group the collection of attribute-specific disposed records. This is done through use of a delimiter-based three-dimensional matrix-based storage architecture. The exemplary architecture enables a four-component, unique natural number based on a numeric-construct i.e., W.Z.Y.X-identifier. These construct identifiers include linear-based, architectural-storage relationships beginning with matrix groups (W) followed by each respective three-dimensional nodal-intercept (Z.Y.X).

The exemplary system establishes attribute-specific records through the use of an expandable delimiter skeleton. This skeleton includes a “Storage Record” (SR) that begins with disposing the respective W.Z.Y.X identifier and a Title respective of an assigned attribute that establishes a “System of Record” (SR). The SR is an authoritative “record” of elements with regard to each attribute that includes a plurality of ontological characterizing static and dynamic elements. These elements replace the static-designs of data-specific fields that typically are the foundation of relational database systems. The exemplary system further enables a “Record-Set” of SR's to be constructed. These SRs are related in storage by a Record-Set specific W.Z.Y.X identifier, that establishes a “Record” comparable to a Legacy Data System (LDS).

The exemplary system also includes the capabilities to relate SRs and thereby respective attributes. This is accomplished by disposing related W.Z.Y.X identifiers within a respective SR that establish W.Z.Y.X relational-connects. This enables the establishment of a delimiter-based “Record Management System” (RMS) comparable to a Relational Database System (RDS) in terms of partitioning, relating and filing attribute records and relating data-specific fields and attributes of function.

The absence of hard drive partitioning and the use of W.Z.Y.X relational-connects in the exemplary system allow for the use of Natural Language Programming (NLP) tools. The NLP tools empower subject matter experts who do not have expert programming skills to program digital based solutions to accomplish their desired workplace functions.

The exemplary system further enables the use of NLP tools by ontologically neutralizing the ambiguities of natural language. This is accomplished by relating all attributes of data and respective datasets to at least one W.Z.Y.X identified attribute of function. This enables ontologically defining data with regard to function. Data to function relationships are programmed through the use of bottom-up, design templates that enable system users to title respective data, datasets and functions. The exemplary system disposes each title into a W.Z.Y.X uniquely identified SR disposed within the RMS. Data to function and function to data relationships are defined within each SR through the use of the relational W.Z.Y.X constructs without the need for expert coding. All attributes of the exemplary RMS are thus ontologically defined. This enables the RMS to provide a parallel operating data system to the existing Legacy data system functions. This approach not only duplicates mission critical functions, but also utilizes the skeleton of expert database designs to template new system functions that include automated information development and outcome-based analytics. Two-way Legacy system and RMS system exchanges enables improved capabilities compared to Legacy data system functions by providing data-driven efficiencies and best practice analytics which were previously unavailable due to the limitations of relational database designs and unconnected data systems.

The RMS common storage-field and SR mediated W.Z.Y.X relational-connects enables connecting SRs of data with SRs of respective data-dependent functions. This is done by using “Data Switch” W.Z.Y.X identifiers that enable “Avatar SR connecting Programs” to “Avatar Connect” SRs with matched DS identifiers. “Avatar-Connects” enable SR-specific two-way element exchanges for Data SRs to “Avatar-Connect” with respective data-driven function-specific SRs. Data SRs transfer their data and thereby activate the “Avatar-Connected” function-specific SR to complete respective programmed data-dependent tasks.

The exemplary system programming is initiated by connecting Legacy systems and data systems of the exemplary embodiments through use of the RMS and NLP tools. The NLP tools enable two-way attribute-exchanges between Legacy systems and RMS data systems. The exemplary arrangement also provides for new Legacy data to be structured within a transport SR, imported into the RMS and “Avatar-Connect”, first with a respective Record-Set and second with a respective Record-Set disposed function-specific SR that completes respective site-specific (Legacy record) programmed data-driven tasks.

The exemplary RMS system interfaces and connects with Legacy data systems by means of paired interfacing SRs for each attribute included in each Legacy data system. The exemplary system includes “Glossary File” of Legacy-specific SRs matching the collection of Legacy data system attributes. The Glossary File is consolidated into a “Master-file” or unique Legacy data system attributes without duplicate representations using W.Z.Y.X mediated relational nodal-connects. Interfacing SRs connect the plurality of duplicate attribute-specific Legacy interfacing SRs, to a single respective interfacing SR of the RMS system that establishes a Nodal-SR. Nodal-SRs route duplicate Legacy attributes using W.Z.Y.X relational connects to other Legacy data systems, and together with the non-nodal interfacing SRs of the exemplary system enable two-way attribute-exchanges between Legacy and the RMS data systems. This enables data from Legacy systems to populate the exemplary system functions and the system of the exemplary embodiment to fuel Legacy data system functions.

The exemplary system utilizes the expandable SR delimiter skeleton and NLP tools to dispose together titles of attributes and respective ontologically characterizing elements. The features also inter-relate other SRs comprising both data and respective data-driven actions that collectively morph a function-specific “static storage-record” into a “Dynamic Storage-Record” (DSR). The DSR provides a defined operational platform to design and administrate respective data and programmed processes to complete assigned functions. DSRs and NLP tools enable user operators to transform their knowledge-driven functions to programmed data-driven functions. The exemplary system thereby establishes a new digital workforce of user operators who do not need expert programming skills. This enables a mobilization of organization knowledge-based assets and enables digitalising data-driven functions of the organization that implements the exemplary system.

The exemplary system establishes a “Virtual Assistant” with regard to each programmed function by enabling new data to activate a respective DSR to complete its respective function. This results in new data-driven efficiencies due to the prior limitations of centrally designed Legacy data systems. Record-Sets of client specific DSRs represent knowledge-directed programming of a user specific “Virtual Assistant” for completing automated data-driven workplace functions. The exemplary system thereby enables transforming a user operator's role from completing workplace functions, to guiding and governing their “Virtual Assistant’ in completing the respective workplace functions enabling increases in user operator productivity.

The DSRs of the exemplary system further enable establishing an Enterprise best practice SR. “Systems of Record” by subject matter experts establishing Enterprise best practice datasets, graded actions and outcomes. This capability also enables defining elements of an application-specific record i.e., data; actions and outcomes, to establish a common-core of data to fuel a peer-network database. Respective Enterprise DSRs enable the establishment of enterprise wide “Systems of Record” i.e., Golden DSRs, and ways for establishing a Record-Set of Enterprise best practice golden DSRs. The exemplary system establishes a peer-network by copying a Golden SR to administrative experts, thereby establishing a common-core “Systems of Record” for each Enterprise function. Administrative experts copy their Golden-SR to site-specific record-sets, where site-specific data fuels respective DSR functions. Such DSR functions generate application-specific records of data; actions and outcomes that fuel site-specific automated information generation and machine learning algorithms that can be used to evolve site-specific best practice. Respective records are further disposed in both the administrative expert's SR and the Enterprise expert Golden-SR for aggregate self-directed analytics and enterprise directed analytics. The records are networked by a common-core “System of Record” thereby completing the knowledge-to-data and data-to-knowledge circle.

NLP tools enable application-experts to modify respective Golden “Systems of Record” based on their best practice workplace knowledge and self-directed analytics, by making aggregate site-specific, machine-learning mediated modifications. The exemplary system thereby utilizes top-down designs to enable bottom-up development of new innovative best practices. Records are identified respective of modifications to assign best practice value records and identify the modifying agent so as to assign value-based compensation.

The exemplary system establishes capabilities for virtual testing that validates regional best practices. This is enabled by the common-core “System of Records” that establishes the collective, peer-based, Enterprise database, and that provides capabilities For validation of best practice modifications prior to implementation and enables guidance and governance of “Virtual Assistants” post activation. Virtual testing also provides the capabilities to support new educational and certification tools for use in the workplace and to provide system training.

The exemplary system establishes means for Artificial Intelligence (AI) and mentor balanced real-time assists to a user operator in completing workplace functions or designing a DSR modification. AI assists are enabled through use of the peer-network that enables the AI function to access a peer-developed enterprise “System of Record” database related to a workplace function to establish a predictive outcome. The mentor-specific assist is enabled by a respective mentor modified “System of Record” based on the mentor-specific knowledge. The exemplary system enables an Avatar-connect between the operator's DSR and the mentor's DSR. This enables using the user's site-specific data to activate the mentor-specific DSR with respective automated actions providing real time recommendations, without the involvement of the mentor. This presents an advantage in widely dispersed brick and motor application sites. Mentor assists can include elucidating the settings that the mentor's experience predicts unexpected adverse outcomes, which would otherwise be difficult to extract from a collective database. Such mentor assists can be used to balance the common database predictive value of AI assists.

This transformational RMS-exemplary embodiment empowers organizations to leverage the expert designs of Legacy data systems to establish a new digital-workforce and provide mobilization of knowledge-based assets. The exemplary system empowers both subject-matter and application experts who do not need to have expert programming skills, to collaboratively transform their knowledge-based, data-driven functions into automated best practice DSRs. The system enables use of “Virtual Assistants” that are guided and governed by peer-networks, enabling new collaborative operational efficiencies and best practices. Collectively, the exemplary system may digitalize all organization functions, idealize Legacy data system functions, and enables organization-wide, guided and governed best practices.

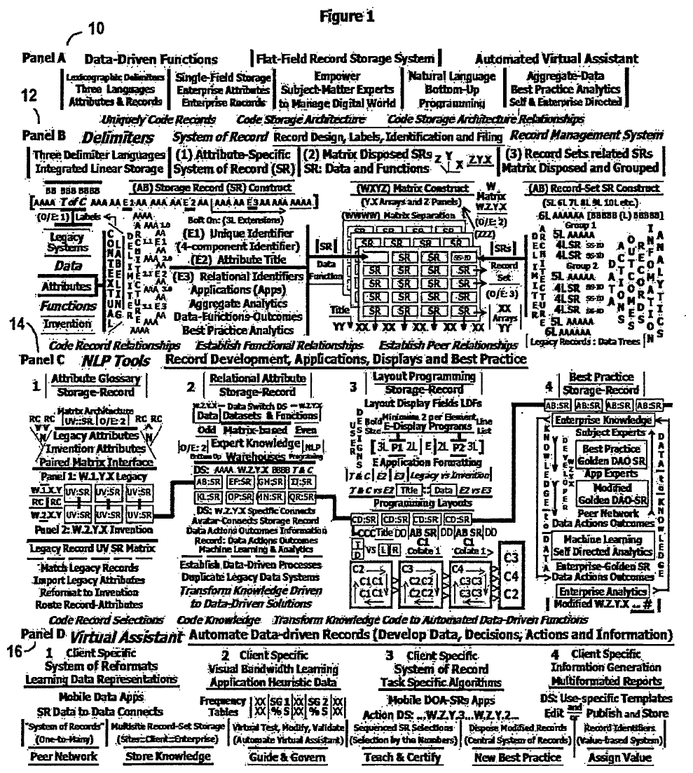

Referring now to FIG. 1, Panel A 10 exemplifies primary system-specific attributes that begin with establishing three lexicographic-based, hierarchal, delimiter languages. These languages provide three separate and complementary hierarchal-delimited flat-field storage-architectures. These storage architectures collectively enable uniquely identifying, grouping, sub grouping and relating, attributes of data and functions. These attributes may be disposed separately and together within the same storage-field. This approach enables matching attributes of a relational database without the need for data-specific fields. Not having data-specific fields eliminates the need for users to have expert-programming skill. This empowers user operators without such expert-programming skills to program data-driven processes using intuitive Natural Language Programming (NLP) tools and bottom-up design templates that intuitively guide the user in establishing datasets and connecting data with respective data-driven generic programs. Knowledge-driven, data-dependent processes are gradually transformed into automated data-driven processes guided and governed by application experts to accelerate and manage the evolving digital world. NLP tools include automated, aggregate-data record-keeping inclusive of data; such as actions and outcomes. This enables process-specific, machine learning algorithms to develop idealized best-practice analytics both self-directed and enterprise directed without the typical type-2 analysis-errors that result from missing data.

Panel B 12 exemplifies Delimiters enabling a “System of Record” (SR). The delimiters are comprised of record-specific designs, labels, identifiers and filings corresponding to each attribute of data and function. The attributes comprise a glossary of Enterprise organization attributes for categories of data in all data systems i.e., legacy and the RMS. Three alphanumeric, lexicographic, hierarchal, delimiter-languages enable integrated linear storage of all attributes within a single storage-field or equivalent. The exemplary system includes a Storage-Record (SR) which includes of labeling and disposing each element that comprises an attribute-specific “System of Record”. This is accomplished by use of the first delimiter language and disposes each SR disposed “System of Record” within a matrix of delimiters. The matrix of delimiters is established using the second delimiter language and produces record-sets of related SRs that use of the third delimiter language. The record sets of related SRs equate with a Legacy system record. Each record-set is disposed within a matrix of delimiters established use of the second delimiter language that groups record sets of SRs from attribute SRs. The language further includes means to uniquely identify each respective matrix disposed attribute-specific SR or record-specific SR by a nodal Z.Y.X matrix identifier which corresponds to a matrix storage site. Collectively these three delimiter languages establish the storage architecture for the exemplary Record Management System (RMS).

The delimiter-enabled, flat-field, storage architectures are illustrated in FIG. 1 respective of the Storage-Record, Matrix and Record-Sets. Collectively these features enable the exemplary embodiment to exceed the functional equivalents of data-specific fields, relational database structures and records included in Legacy data systems.

The exemplary system assigns a numeric identifier (W) with regard to each matrix to assign matrix-specificity respective of each Z.Y.X identifier. This establishes a unique W.Z.Y.X identifier that enables the establishment unlimited Matrix groups separated and connected in flat-field storage by WWWW delimiters. Matrix-specific flat-field storage is unlimited through the use of indefinite numbers YY lines indefinite numbers of XX arrays comprising unlimited numbers of panels disposed by ZZZ delimiters. The exemplary matrix record system thereby enables unlimited numbers of attributes grouped, sub grouped and sub-sub grouped by means of panel, line and column delimiters i.e., ZZZ, YY and XX, respectively.

Delimiters for establishing an SR or a Record-Set of SRs are comprised of Lexicographic character strings i.e., 2L: AA, 3L: AAA, 4L: AAAA, 5L AAAAA, etc. Each string is bracketed by a digital empty-space. The space is used to enable hierarchal grouping and sub grouping of disposed elements by bracketing each disposed element within respective linear-disposed, hierarchal delimiters.

Exemplary SR constructs are comprised of 2L, 3L and 4L delimiters. Record Sets begin with 5L to bracket 4L bracketed SRs. Increased string-lengths provide for disposing 4L attributes categorically grouped, sub grouped and sub-sub grouped etc. by means of Enterprise designed delimiter templates. The delimiter templates provide uniform record filings across the Enterprise enabling efficient search and retrieval tools.

Linear disposed hierarchal delimiters bracketing respective elements establish a telescoping hierarchal “Storage Architecture” as illustrated for both an SR (right) and a Record-Set of SRs (left).

The RMS utilizes three delimiter-based, odd and even pairings. These pairings include odd and even character-mediated delimiter-pairings (O/E: 1), odd and even numbered panel-mediated SR-pairings (O/E: 2) and odd and even numbered line-mediated attribute-pairings (O/E: 3).

O/E: 1 character pairings exemplified as (AB,) enable matched odd and even character delimiter strings used for matched delimiter constructs, one composed of the odd character and the other composed of the even character. The exemplary system utilizes this pairing to develop matched delimiter constructs comprising an SR, with the odd construct disposing elements and the even construct disposing respective matched labels. The matched labels provide a “Table of Contents” (T of C) respective of each SR “System of Record”. Unmatched characters enable non-hierarchal storage through use of string length. With regard to an SR, the exemplary system utilizes this difference to dispose the even character T of C to follow the 4L that initiates an SR construct and with regard to a record-set of SRs, utilizes this difference to dispose a categorical label that defines respective groups, sub groups and sub-sub groupings of SRs comprising the record-construct. Non-AB character pairings enable grouping function-directed sets of both SR attribute constructs and Record-Set SR constructs.

Both SR and Record-Set delimiter constructs are extendable without limitation by disposing elements and SRs by means of the [3L 2L E 2L 3L) storage-moiety construct respective of an SR construct and by means of the 4L SR comprising a Record-Set, respectively. The SR based [3L 2L E 2L 3L) storage-moiety is extended by replacing the last 3L bracketing delimiter with the next [3L 2L E 2L 3L) storage-moiety i.e., [3L 2L E 2L 3L 2L E 2L 3L] and the Record-Set is extended by replacing the last 4L of a [4L Attribute 4L) with the next [4L Attribute 4L) storage-moiety i.e., [4L Attribute 4L Attribute 4L]. Hierarchal record-set storage of SRs is used to provide a SR hierarchal construct respective of a Legacy record of attributes as well as a construct an attribute decision tree.

The 3L 2L E 2L 3L storage moiety enables a Y.X panel-construct comprised of 3L initiating (Y) lines and 2L initiating (X) columns. This provides the capability to uniquely identify each disposed element by a Y.X intercept respective of the Y-based and X-based arrays of respective 3L and 2L delimiter constructs i.e., [3L 3.0 2L 3.1 2L 3.2 2L 3L 4.0 2L 4.1 2L 4.2 21 4.3 2L 3L 4L]. A plurality of 2L-disposed E1 and E2 components enable multiple function-specific identifiers and Titles with regard to each 4L attribute of SR expanding applications through decentralized function-specific programming.

The composite structure of SRs as an attribute-specific attribute or as a record-set of SRs enables maximum portability of attributes. The exemplary structure further enables assigning generic data and action programs to a disposed SR identified by respective bracketing 4L delimiters that mark the beginning and end of disposed elements of an SR-specific “set of records” that fuel an assigned SR-specific generic data program.

SR portability and expandable “records of elements” enables a generic SR that is centrally developed, to be transported to decentralized locations with means for customization of the respective “records of elements” to idealize a multitude of regional applications.

The SR construct requires a minimum of two elements i.e. E1 and E2 respective of the W.Z.Y.X identifier and Attribute-Title, respectively. Additional elements represented as E3s include attribute-specific elements, and other SRs constructed of unmatched odd/even characters comprising their respective 4L, 3L and 2L SR delimiters to achieve non-hierarchal storage within the parent or host SR. SR storage within an SR enables disposing functional attributes supporting an operational platform that includes means for data storage, data-dependent action-specific programs, outcome storage, record storage respective of data, actions and outcomes, best practice analytics, information generation and other means for a 4L SR portable App.

The second language of delimiters establishes a hierarchal, matrix-based, storage-architecture. This architecture disposes individual SRs or Record-Sets of SRs grouped by means of Matrix (W) separation, sub grouped by means of matrix disposed panels (Z) i.e., tables, and sub-sub grouped by unlimited arrays of delimited lines (Y) and columns (X) comprising extendable tables. The architecture further includes means for establishing a four-component unique numeric-identifier natural number construct i.e. W.Z.Y.X respective of each Z, Y and X nodal-intercept, identifying a respective matrix-disposed storage site. This uniquely identities each respective disposed storage-record by means of disposing the respective unique matrix-identifier within each attribute-specific SR or each record-specific SR comprising a record-set of SRs.

Panel C 14 illustrates with heavy lines the linear-construct of delimiter-mediated connects of all SRs and SR-mediated functions of a single storage-field or equivalent, managed by the exemplary transformational RMS. Four applications of NLP tools that utilize storage-record (SR) constructs to enable record development, applications, visual displays and best practices are illustrated. The first application establishes a glossary of Invention SR “Systems of Records” respective attributes of data and function, with capabilities to import Legacy system data and export RMS data and Information. The second application establishes an SR operational platform that connects attributes of data and programmed actions to complete a workplace function. The second application further includes means to automate respective functions through use of imported Legacy data. This establishes an automated, parallel operating Legacy data system that duplicates critical Legacy functions and frames RMS functions. The third application enables duplicating all Legacy Layouts by means of a single generic programmable RMS Layout. The fourth application establishes a peer network of collaborators to define best practice and establish new best practices.

The first application establishes a “Glossary of Attributes” with respect to each Legacy data system and the RMS. The first application provides (O/E: 2) SR panel pairings that establish a “Master File” by means of W.Z.Y.X connects, respective of unique Enterprise attributes of data and functions. The first application nodal connects all Legacy data systems to the exemplary system by enabling two-way exchanges which provide for duplicate Legacy data to be shared across respective system nodal-networked Legacy data systems.

The second application provides functionality to relate SRs by disposing within an SR, an E3 relational construct of W.Z.Y.X identifiers with regard to one or a plurality of related SRs. Matrix line pairings (O/E: 3) enable data-specific SRs to be disposed within odd-line matrix arrays and related functions to be disposed within a sequenced even line matrix array with odd and even line relationships. SR to SR relationships are documented within each respective SR through E3 relational W.Z.Y.X constructs. Line-pairings enable subgrouping respective of function-specific SRs within matrix panels.

The third application establishes a generic programmable layout comprised of multitudes of programmable display fields, paired with regard to displaying data and respective labels, Display fields are Y.X. connected to DD disposed SRs. This functionality is a generic CD-SR display program. Display fields are positioned respective of each other by collating and positioning fields that store coordinates within respective DD disposed SRs, collectively establishing a layout design with all process enabled by means of NLP tools, and without data-specific layout fields. Each CD program is disposed within a Legacy data system specific matrix panel that is uniquely identified by the W.Z.Y.X identifier and titled respective of the Legacy system and layout. Current application of a CD program replaces old SR disposed data with current data with regard to matched W.Z.Y.X identifiers subsequently displayed as programmed.

CD-based designs enable each client-operator to idealize visual based learning with function-specific programmed layouts that support best practice decision-making and enable a single data set to be displayed differently across a plurality of client users.

The fourth application exemplifies the portability of SR constructs supporting peer-networks of application-experts that begin with best practice SRs and modify SRs by developing new best practices. Function-specific, best practice operational SRs are initially developed by subject matter experts to generate records of application-specific data; actions and outcomes i.e., knowledge-to-data Golden-SRs, to establish a record set of enterprise best practices. Portability enables transferring a respective Golden-SR to a plurality of application specialists. They in turn transfer a plurality to record-sets of individualized applications. Respective records support site-specific machine learning, and portability enables disposing each application record within the respective SR of the application expert, and the respective enterprise best practice SR is used to fuel best practice analytics respective of each application expert and the enterprise i.e., data-to-knowledge.

Panel D16 exemplifies the primary elements that enable establishing a “Virtual Assistant”. The virtual assistant may be used for automating data acquisition, decision making, parameter graded actions and the development of use-directed information by means of four primary NLP tools. The NLP tools include processes of reformatting data to enable mobile data acquisitions (1), idealizing visual bandwidth learning respective of displaying together heuristic and selected data sets coupled to comparative frequency-tables graphs etc. (2), establishing parameter-graded actions enabling mobile completions of programmed tasks (3) and generating a plurality of client specific and use-specific informative reports (4).

Exemplary Virtual Assistant development begins with each user operator copying a function-specific Expert DOR “System of Record” to a user-specific knowledge-based record-set of DOR functions. This establishes a common “System of Records” across user-operators and can be further disposed by user-operators to site-specific record-sets for automation with regard to site-specific data. The user disposed DOR can be used to manually operate the DOR with regard to site-specific data to complete workplace functions, while developing a record set of user-specific, site-specific, application-specific, records of data, actions and outcomes. The specific records can be used for comparison to respective matched virtual-records with regard to Expert data-driven programmed actions and Artificial Intelligence (AT) predicted outcomes to establish virtual testing. Automated comparison charts enable validation by the numbers, enabling the user-operator to either accept the Expert DOR mediated “Virtual Assistant” or modify by adding respective data, actions and outcomes to achieve user-specific, application-specific best practice while maintaing common-core elements. The modified DOR is uniquely identified as are subsequent records of respective modified elements, to include AI-mediated virtual-outcomes respective of modified data and actions. AI mediated Expert-DOR virtual-outcomes are used for comparative analytics by the numbers to complete virtual-validation of modified DORs. This enables activating the DOR for automated data-driven applications that establishes the client-specific Virtual Assistant. DOR-specific, attribute selection templates enable establishing consistency-standards by the numbers for sequencing attribute selections in completing a respective DOR mediated function. This may be of particular value in teaching and certification endeavors. These same virtual-actual record comparisons post automation enable user-operator mediated guidance and governance of “Virtual Assistants”. Disposing the same records within the central Expert-DOR enables post-automation Enterprise-mediated guidance, governance and identification of new regional developed best practices. This capability for disposing the W.Z.Y.X identifier respective of each user-operator within each modified record, enables assigning value-based purchasing and/or reimbursement respective of client-operators.

The exemplary embodiment enables each user operator to evolve a “Virtual Assistant” for automating their respective knowledge-driven, data-dependent, workplace functions by completing such workplace functions within the exemplary system. The exemplary embodiment enables both non-linear and linear based “Pattern Analysis Learning” to support work-mediated programming of client-specific DOP-SRs. This is done by the numbers with “non-linear based learning” enabled by means of a W.Z.Y.X-mediated identifiers uniquely identifying each component comprising the user specific DOP-SR designated “System of Record” and “linear based learning”. Linear based learning is achieved by identifying user-specific sequenced application of respective elements of the DOP-SR sequenced hierarchal delimiter structure. Peer networks of Application Experts established by Golden DOA-SRs, enable each of the respective user-specific “Pattern Analysis Learning” templates to fuel Enterprise-specific “Pattern Analysis Learning” means for Enterprise best practice analyses by the numbers. This further fuels development of Enterprise-specific, Artificial Intelligence (AI) algorithms supporting real-time AI-assists.

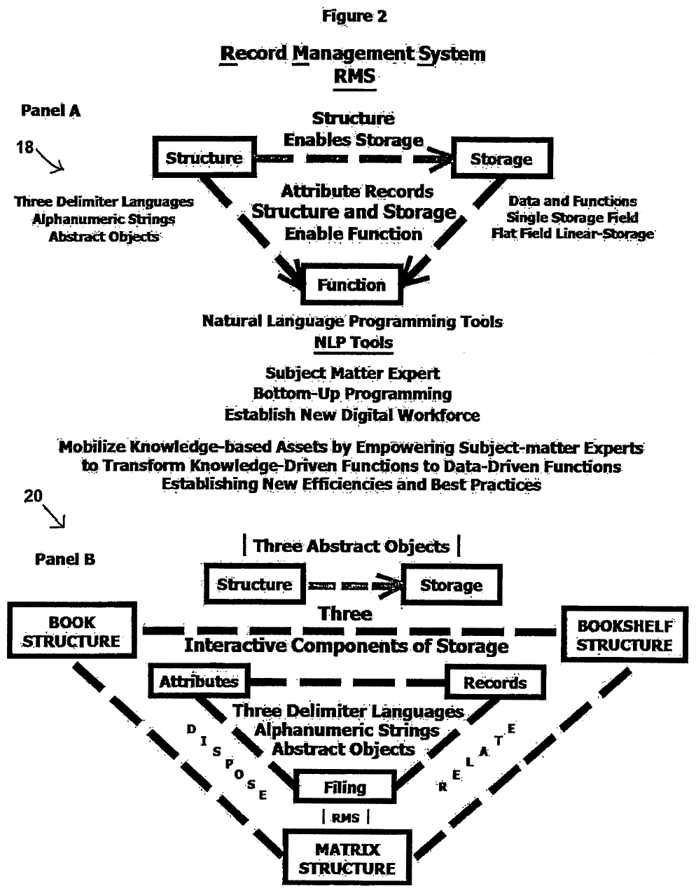

FIG. 2 schematically represents the exemplary Record Management System (RMS). The RMS uses three rule-based, alphanumeric delimiter languages that establish a consubstantial relationship between Attribute Structure, Attribute Storage and Attribute Function represented in Panel A 18. The rule-based, delimiter storage languages establish abstract, object-related, storage-structures that enable flat-field, linear-storage of all elements that comprise attributes of data and function. The languages further provide the capabilities to store these respective elements separately and together within a single storage-field or equivalent without the physical partitioning of data-specific fields. The structure-storage relationships of the exemplary system collectively enable the use of Natural Language Programming (NLP) tools. These tools empower both Subject-matter experts and Application experts absent expert who do not have programming skills, to transform their knowledge-driven workplace functions into automated, data-driven workplace-functions that are guided and governed by these design experts. This enables using these capabilities to establish a new digital-design and management workforce. The three-abstract storage-structures comprising the RMS and are descriptively labeled in Panel B 20 of FIG. 2, as a Book-Structure, a Bookshelf-Structure and a Matrix-Structure that skeletonizes the storage of Attributes (Books) and Records of attributes (Bookshelves). The approach also establishes a numeric-based, W.Z.Y.X filing system that enables uniquely identifying, relating, storing and retrieving each respective disposed book and bookshelf structure comprising which makes up the exemplary data system.

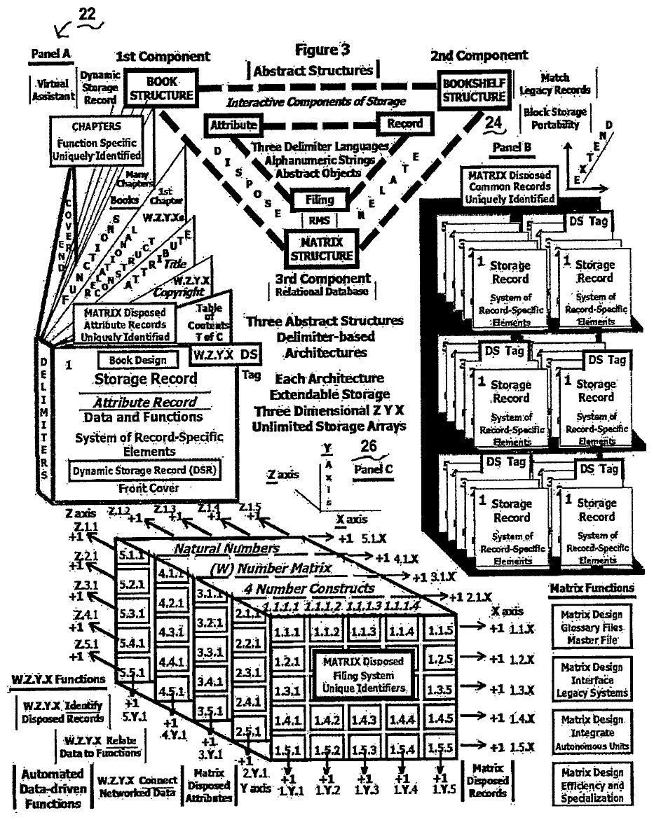

FIG. 3 illustrates the three-abstract storage-structures i.e., a Book-Structure, a Bookshelf-Structure and a Matrix-Structure i.e., Panel A 24, Panel B 24 and Panel C 26, respectively.

The Book-Structure establishes an attribute-specific Storage-Record (SR) i.e., “System of Records”. It is based on the plurality of elements that collectively identify, title, and characterize an attribute of either data or function. Respective attribute-specific elements are grouped and sub grouped within hierarchal delimiters which include function-specific page and chapter equivalents. The disposed bracketed collective is further bracketed within related hierarchal delimiters i.e., “Book. Covers”. This enables all attributes comprising the exemplary data system to be stored separately and together within a single storage-field or equivalent. Each disposed attribute-specific element is defined by a “Table of Contents” (T of C). The T of C is constructed of hierarchal matched and character mismatched delimiters with regard to the book structure delimiters. The T of C disposes element-specific labels within respective T of C hierarchal matched delimiters to label and locate each disposed attribute-specific element bracketed within the book covers. Attribute elements include a minimum of an attribute-specific, W.Z.Y.X unique-identifier i.e., Copyright (Page-1), an attribute Title (Page-2) and the unique W.Z.Y.X identifier or identifiers respective of related attributes (1^{st} Chapter) i.e., data-specific W.Z.Y.X identifiers disposed within the 1^{st} Chapter of related data-driven functions and function-specific W.Z.Y.X unique identifiers disposed within the 1^{st} Chapter of each related function-specific data. Thus the exemplary arrangement ontologically defines data respective of a related function and defines function respective of related data thereby neutralizing the inconsistencies of natural language absent informative text that enables Natural Language Programming (NLP) tools.

Attribute-specific storage of W.Z.Y.X-specific coded relationships without the need for computer code produced by skilled programmers enable untethered portability of attributes and empowers subject matter experts to relate data and function via NLP tools. The book-structure is extendable by adding function-specific chapters that morph a static Storage-Record into a Dynamic Storage-Record (DSR). A DSR is capable of completing chapter-disposed programmed functions, thereby establishing the building block of the data-driven system functions. The DS-Tag is a “data switch” comprised of W.Z.Y.X identifiers that enable the system to “connect” related SRs disposed separately and together within the same storage-field or equivalent. This capability provides for a “Connect” to activate SR-specific programs that enable the transfer of data from a respective Data SR to a respective related Function SR. This can activate the function SR to complete the respective programmed function. The DS establishes regional control of respective functions by activating or inactivating a respective Function-specific SR.

The abstract bookshelf-structure groups and sub groups multitudes of storage records i.e., books (Panel A 22) that constitute a Record of related Storage-Records with block groupings enabling block transfers that maximize Record portability.

The abstract matrix-storage structure is comprised of multitudes of delimiter-based matrix-panels. Panels include multitudes of delimiter-based lines and columns to dispose SRs and Records of SRs which may include unlimited panels which make up a matrix and unlimited line and column extensions comprising a respective panel. The abstract matrix-structure further elucidates the W.Z.Y.X numeric-based matrix filing system with the capabilities to uniquely identify each respective disposed SR or record-specific collection of SRs.

The matrix skeleton provides an incremental, natural number based, filing system respective of the Z (panel), Y (Line) and X (Column) three-axis structural-intersect with the matrix construct (W) completing the unique four-component W.Z.Y.X identifier. W.Z.Y.X functions include uniquely identifying, relating and connecting SRs that collectively enable automating data-driven functions. Matrix designs enable the exemplary system to establish glossaries of attributes of each legacy data system, and to collate the plurality of Legacy glossaries into a master attribute-file absent the duplicity of legacy data systems. The matrix system further establishes the capability for the Master-file to interface with Legacy data systems to route attributes between previously unconnected legacy data systems. This capability may be used to eliminate duplicate data entries and functions across a plurality of Legacy data systems as well as to import and export attributes between the exemplary system and the connected legacy data systems through use of a DS mediated connecting-network. Importing data per the network of DS W.Z.Y.X identifiers establishes a channel to fuel the exemplary systems automated data-driven functions that include automated information generation respective of data, actions and outcomes, and to export respective information and outcomes to the respective Legacy data systems.

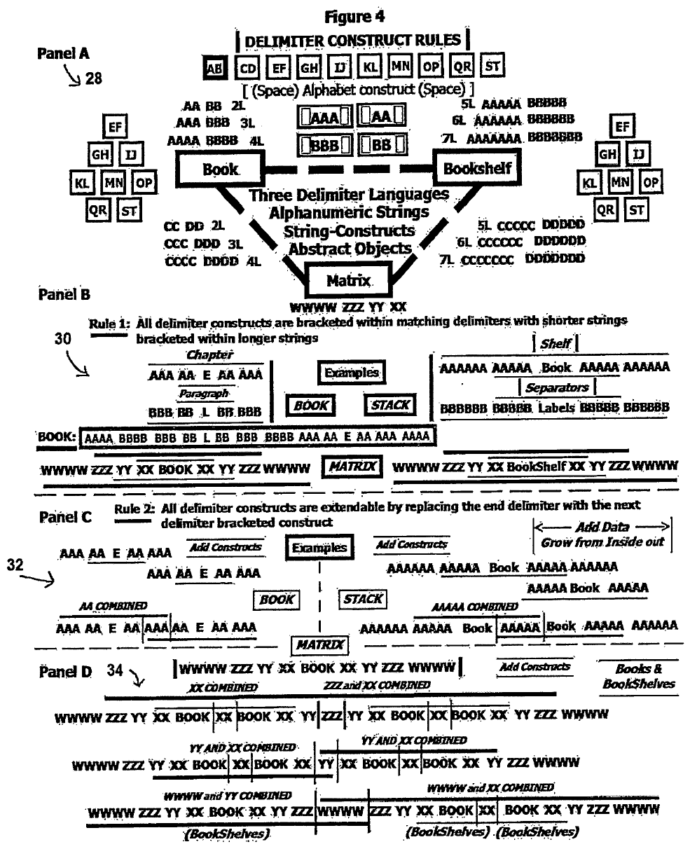

FIG. 4 schematically illustrates the delimiter constructs that comprise the three delimiter-based storage languages, and the structuring Rules that are used to generate the abstract storage structures of FIG. 3.

Panel A 28 illustrates the three sets of alphanumeric hierarchal delimiter constructs. Panel B 30 illustrates the three language-specific delimiter constructs that include the components to construct the three storage designs. Panel C 32 illustrates the construction processes that establish the abstract storage designs respective of the Book and Bookshelves. Panel 1) 34 illustrates the respective matrix construction processes that establish the matrix that disposes respective Book and Bookshelf designs.

Panel A 28 illustrates 10 odd-even alphabet pairings from A to T corresponding to Book and Bookshelf constructs, and characters W, X, Y, and Z corresponding to Matrix constructs. Character strings are bracketed by digital spaces to complete each respective delimiter construct, that enables object-based separation within shared field storage. Book-specific delimiters are comprised of two, three and four-character strings i.e., 2L, 3L and 4L respectively. Bookshelf-specific delimiters are comprised of greater than four-character strings i.e., 5L, 6L, 7L . . . respectively as illustrated in Panel A 28. The exemplary system enables a minimum of 10 sets of hooks and bookshelf designs that enables character-specific programs and thereby character specific functions and applications, within the system. Matrix-specific delimiter constructs are comprised of WWWW, ZZZ, YY and XX character strings bracketed within blank spaces are also illustrated in Panel A 28. These enable flat-field disposition of any character-based Book or Bookshelf construct absent string length or character based confused storage. Two delimiter construct rules govern all three delimiter languages with regard to establishing the “base-constructs” (Rule 1) Panel B 30 and utilizing the respective “base-constructs” to establish the abstract record designs that comprise Books, Bookshelves and Matrices (Rule 2) Panels C 32 & D 34.

Panel B 30 illustrates odd and even character paired delimiter constructs used in assembling book-constructs and bookshelf-constructs. These are used to dispose book-elements and books respectively within odd character constructs. The respective labels of book-elements and books are included within matched even-character constructs. Matrix constructs comprised of WWWW, ZZZ, YY and XX delimiter constructs dispose books and record-specific groups of books within XX column delimiters, disposed within YY delimiter constructs comprising ZZZ panel constructs.

Rule 1 brackets each respective elements of a book within 2L delimiters establishing “paragraph-constructs,” and then brackets the 2L bracketed “paragraph-constructs” within matched 3L delimiters establishing “Chapter-constructs”, which include the “paragraph-constructs”. Bracketing the collective of “paragraph-constructs” within the character matched 4L delimiter-constructs establishes a respective abstract “Book” construct. Rule 1 brackets “Book” constructs within 5L delimiter constructs, and then within 6L delimiters with each successive set of bracketing delimiters establishing a new respective grouping. In accordance with Rule 1 matrix-constructs dispose books and bookshelves, by means of bracketing each book-construct or each bookshelf-construct within XX matrix-column delimiters, which are bracketed within YY matrix-line delimiters. The XX, YY delimiters are bracketed within ZZZ matrix-panel delimiters and panel delimiters are bracketed within WWWW matrix delimiters as illustrated Panel B 30. In summary, application of Rule 1 with respect to book and bookshelf delimiters are string length based, and with respect to the matrix delimiters are character-based.

Rule 2 in Panel C 32 establishes the capability to extend the delimiter bracketed constructs established by Rule 1. This is done by replacing the end bracket delimiter with a bracketed delimiter-construct comprised of delimiters matched to the end delimiter being replaced. Panel C 32 illustrates the “condensing process” respective of book-constructs on the left and respective of shelf-constructs on the right. Panel D 34 illustrates the Rule 2 “condensing process” with regard to extending matrix storage designs. The Rule 2 “condensing process” enables unlimited extensions of the abstract structures comprising Books, Bookshelves and Matrix designs.

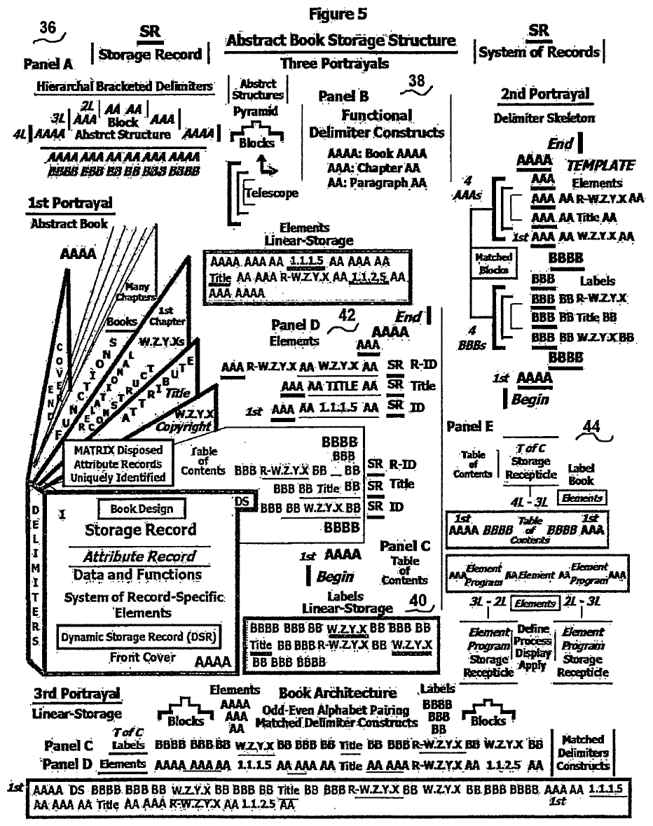

FIG. 5 illustrates key components of an exemplary Storage Record (SR) i.e., A “System of Records” (SR) with 4L, 3L and 2L hierarchal delimiters (Panel A 36) Functional Delimiter constructs are illustrated in (Panel B 38) comprised of Book covers, chapter borders and paragraph borders. This FIG. 5 further illustrates three portrayals of a Storage-Record's “System of Records”. The first portrayal illustrates the functional components i.e., the Abstract Book Architecture of FIG. 3 comprised of a minimum of three elements i.e., unique Identifier labeled W.Z.Y.X, a Title labeled Title, and the unique W.Z.Y.X identifiers of related attributes labeled R-W.Z.Y.X that ontologically define each attribute of data respective of a related function. The second portrayal illustrates the hierarchal telescoping delimiter skeleton of matched 4L, 3L and 2L blocks (4 AAAs) respective of elements and labels comprising a “System of Records”. The third portrayal illustrates linear-storage of the matched element and label delimiter odd-even character constructs, respectively with the Label Construct (Panel C 40) disposed between the first 4L and the first 3L of the element construct (Panel D 42). Matched delimiter blocks enable matching a respective disposed label to a matched disposed element for each element comprising the “System of Records”. Panel E 44 elucidates how the exemplary embodiment utilizes the mismatched delimiter interfaces i.e., 4L-3L that dispose the T of C to define the SR Elements and further dispose element programs between 3L-2L and 2L-3L interfaces juxtaposed with the respective 2L bracketed element. Most important the second portrayal illustrates unlimited capabilities for the exemplary embodiment to add 3L Chapter constructs and unlimited capability to add 2L bracketed columns of data respective of each 3L Chapter program.

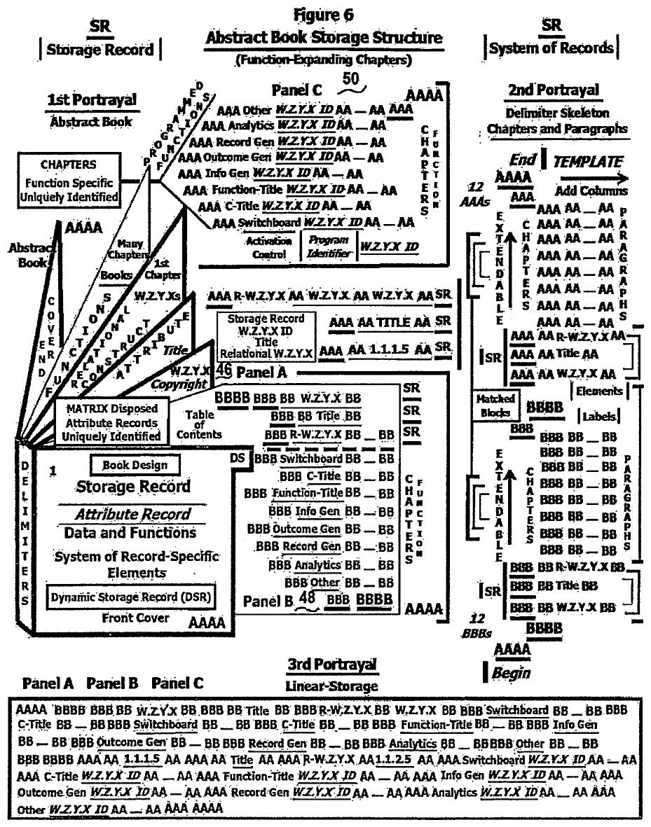

FIG. 6 illustrates the three portrayals of features in FIG. 5 with each expanded to include Functional Chapters that transition a static non-functioning SR respective of a Titled Function that does not include function-specific chapters, into a Dynamic Storage-Record hosting automated data-driven processes. The defining SR composite of W.Z.Y.X, Title and R-W.Z.Y.X elements are labeled SRs in Panel A 46 and Chapter-based programs are illustrated in Panels B 48 & C 50 respective of labels and chapter programs. Chapter-specific 2L disposed elements include data, intermediates, results etc. (Panel C 50) and are labeled within the block matched “Table of Contents” Panel B 48.

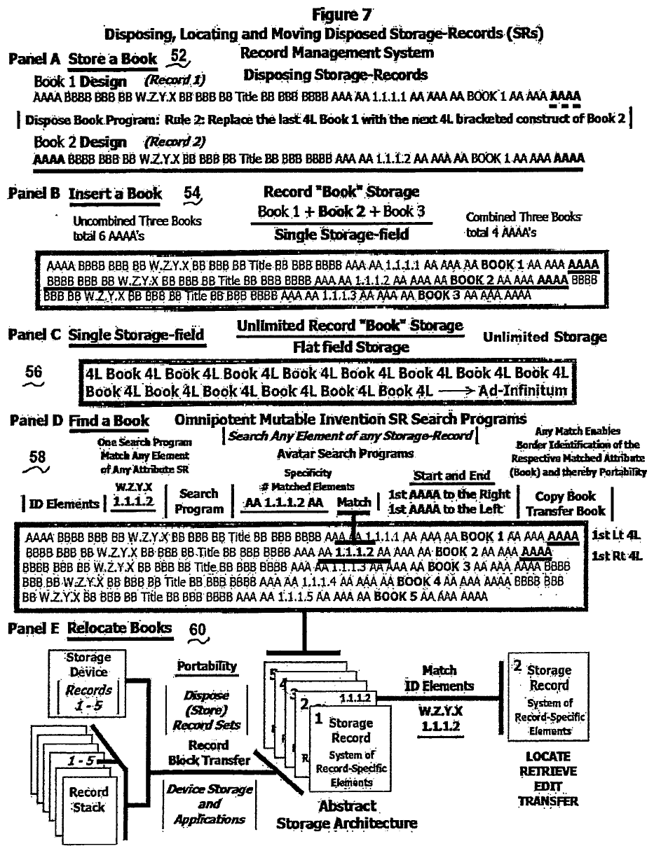

FIG. 7 illustrates how the exemplary arrangement Disposes, Locates and Moves respective disposed Storage-Records (SRs) i.e., “Systems of Records” with respect to Legacy and non-Legacy Records and attributes thereof which, collectively make up the Enterprise data system disposed within a single storage-field or equivalent. Panel A 52 illustrates “Storing an SR Book” by means of Rule 2 by which mediated sequenced connects that replaces the end 4L delimiter with the next 4L bracketed SR. Panel B 54 illustrates “Inserting a Book” i.e., SR Storage Record within a disposed string of 4L SRs (Books) by means of the same Rule 2 mediated 4L substitution. Rule 2 mediated SR storage means for unlimited single field Book storage (Panel C 58) without the hard-drive partitioning of data-specific field storage that enable the exemplary system “Omnipotent Mutable SR “Avatar” Search Programs” represented in Panel D58. A single Avatar “Search” Program is capable of “Matching” any Element of any SR Attribute (Book), and further provides for “Any Match” to enable 4L border identification and thereby SR-specific applications and portability. A W.Z.Y.X programmed “Match” is Illustrated in Panel D 58 noting that SR search specificity increases with the number of elements matched respective of duplicity in single-field disposed SRs without use of attribute-specific Storage fields. Panel E 60 exemplifies the portability of SRs enabling site-specific modifications and applications that may include Device storage and applications.

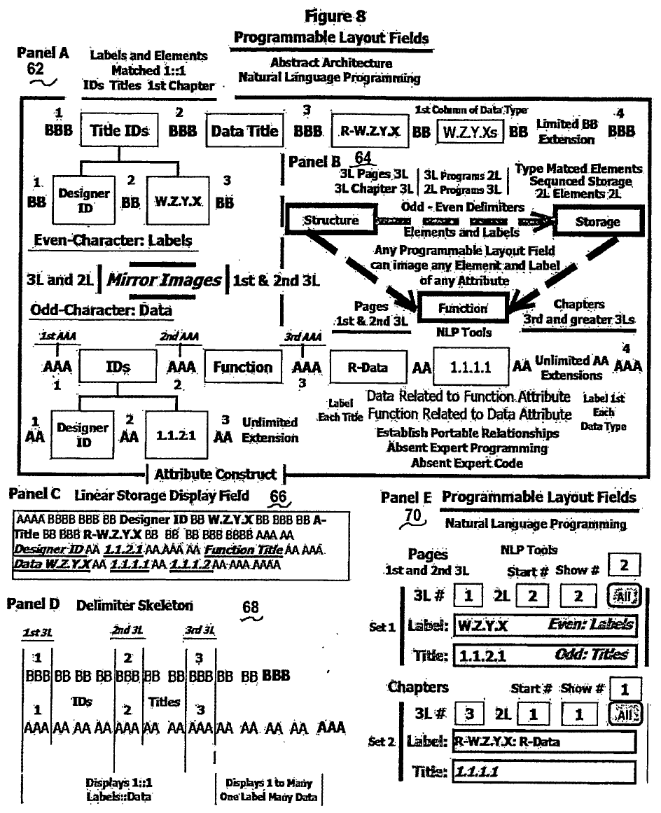

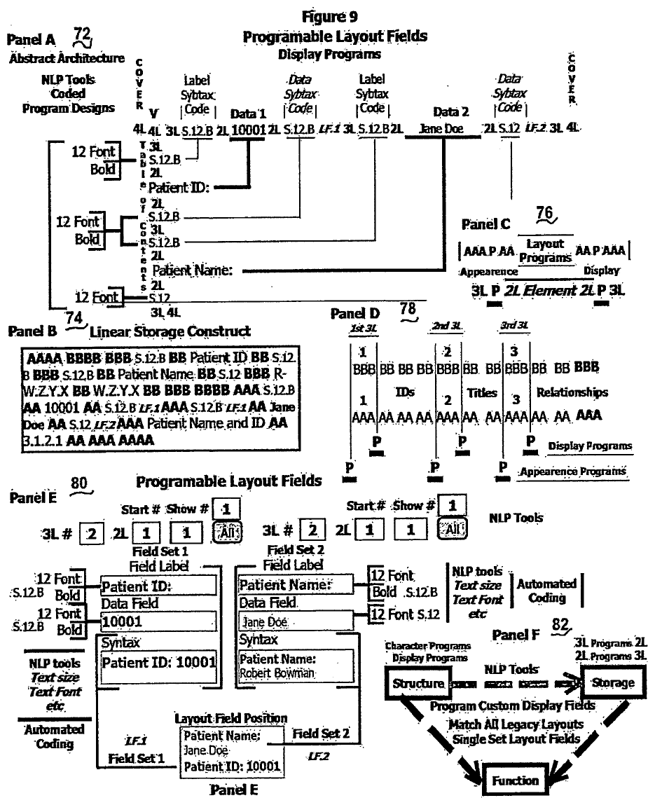

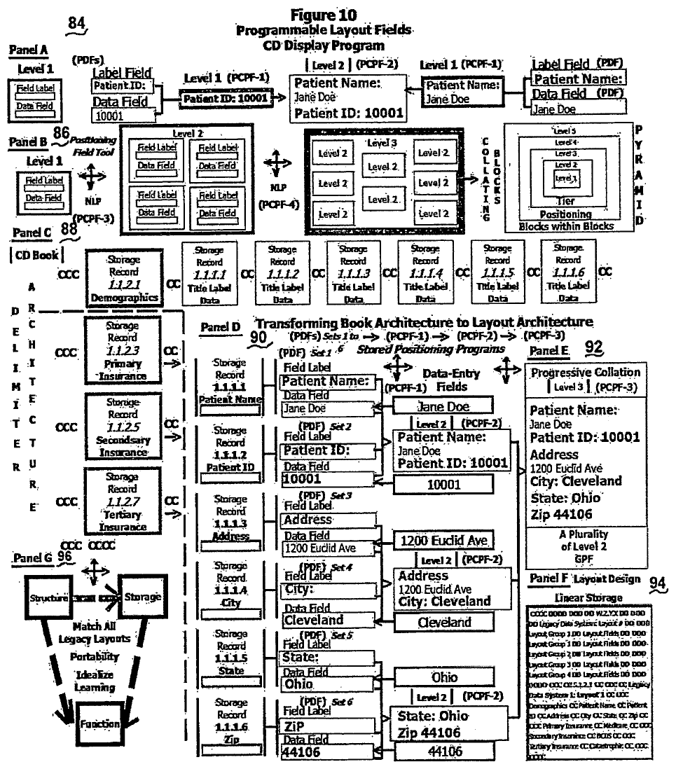

This exemplary arrangement enables matching all Layout designs of Legacy data systems by means of a single set of programmable Layout-Fields with means to match field content (FIG. 8), match the visual presentation of field content (FIG. 9) and match Layout designs (FIG. 10. The exemplary system further includes the capability to program respective programmable Layout-Fields by means of NLP tools without expert programming skills or code, along with the capability to dispose a Layout-specific program within a 4L Storage-Record Book format establishing portability for site-specific applications free of central database programmed tethers. In addition, programmable Layout-Fields enable developing layouts that are data-specific, user-specific and application-specific to maximize visual bandwidth learning for each user and each application thereof, with portability of a “Layout Program” to load into devices operating independent of a central database connection.

FIG. 8 illustrates the capabilities of the Natural Language Programming NLP tools of the exemplary embodiment to assign field content to “Programmable Layout Fields” enabled by the defined delimiter structure of each Storage-Record (SR) illustrated within Panel A 62. Panel A 62 illustrates the structural, storage and functional differences of the first and second 3L delimiters exemplified as AAA and BBB, compared to the third or greater 3L delimiters i.e., abstract architecture respective of “Pages of the Book with Titled Text” compared to “Chapters of the Book with 2L bracketed Paragraphs”. Attribute-specific IDs and Titles must each be labeled to enable each assigned function. Therefore the exemplary system enables matching (mirroring) odd and even character 3L and 2L hierarchal sequences to label each respective disposed element within the first and second 3L bracketed subgroups. The exemplary system labels each Chapter Title disposed between each 3L and first 2L delimiters beginning with the third 3L delimiter of each Storage-Record as illustrated. However, the exemplary system labels only the first element of each respective type-matched sub grouping of elements which make up a “Paragraph”. Sequenced subgroupings of each set of type-matched elements collectively make up the “Paragraphs” of a Chapter Group.

Panel B 64 illustrates the structural and storage relationships illustrated in Panel A 62 that collectively enable a single programmable Layout-Field of the system to visualize and label any Element of any Attribute within the exemplary data system. Panel C 66 illustrates linear storage of the abstract delimiter structure in Panel A 62. Panel D 68 illustrates the delimiter Skeleton advanced in Panel A 62 noting the one to one and one to many functional designations used in labeling Attribute-specific elements. Panel E 70 exemplifies the NLP tools that enable assigning attribute elements to a respective set of programmable Layout-fields visualizing the disposed Labels and respective Element.

FIG. 9 schematically describes the capabilities of the exemplary system to establish Layout-Programs which utilize each set of Programmable Layout-Fields to control the visual-bandwidth and application of Layout-specific elements. Labels and Title positions are programmable respective of each other's position and similarly these programmed pairs are programmable with regard to each other's position. Panel A 72 illustrates the abstract architecture that enables the system to utilize the 3L-2L and the 2L-3L interfaces created by the 2L block disposed within the 3L block of the Attribute Storage-Record SR. Panel B 74 illustrates the linear-storage construct respective of the example or Panel A 72. Panel C 76 illustrates the delimiter skeleton of a single element that is further illustrated by means of the Attribute delimiter skeleton in Panel D 78. The NLP tools and the means to establish the respective coded Layout-specific programs respective of each set of Programmable Layout-Fields are illustrated in Panel E 80. The system further includes the ability to position Labels with respect to matched elements by means of a Layout-Field Positioning Field. Panel F 82 relates the structural programs controlling attribute specific visual bandwidth and displays, with the functionality to dispose them absent expert programming. This capability enables the exemplary system to match each Attribute-specific Layout display with those of Legacy data systems.

FIG. 10 further exemplifies the collating “block in block” storage architecture of the exemplary system that completes the systems display capabilities for providing the capability to duplicate any Layout of any Legacy data system. This is accomplished through use of a single Layout of Programmable Generic Layout Fields including Programmable Display fields (PDF) and successive tiered Programmable Collating and Positioning Fields (PCPF). FIG. 10 further demonstrates the capability to dispose a respective Layout-design within a CD delimiter 4L Book architecture to enable storage, retrieval and portability respective of any Layout design enabling device-mediated applications independent of the RMS data system, with new data formatted for import into the RMS data system. Panel A 84 illustrates Level 1 and Level 2 programmed processes directing labeling, collating and positioning tools respective of the system's block storage collating capabilities. Panel B 86 exemplifies the successive “Block in Block” pyramidal expansion of a respective Layout-Design collating the tiered PCPFs into a single Layout field exemplified as Level 5 within the collating Block illustration of Panel B. Panel C 88 illustrates the CD delimiter book architecture that enables storage of respective PDF and PPF programmed Layout designs.

Panel D 90 illustrates Level 1 and Level 2 successive tiered block reconstruction tools respective of the CCC Demographics Chapter guided by CD disposed programs stored consequent to the initial layout design. Panel E 92 illustrates the Level 3 block of collated data respective of Chapter 1 (Demographics) and Panel D 90 programmed progressive tiered reconstruction processes. Panel D 90 and Panel E 92 processes are subsequently repeated for each respective chapter with a CCC chapter-collating tier completing a respective Layout design. Panel F 94 exemplifies linear-storage of the CD disposed Layout design and Panel G 96 illustrates that the respective linear disposed CD delimiter Architectural structure that enables CCC grouping and CC sub grouping of respective Layout-specific field content and field positioning.

Sequenced chapter storage enables sequenced chapter-specific reconstructions followed by positional tiered chapter-specific layout constructs means which enables the exemplary system to match Legacy data system layouts by means of a single layout of programmable Layout Fields. In addition, user-specific customization of data display layouts can enhance user-specific learning, supporting more efficient and improved decision-making. Further, CD book storage enables portability and reconstruction facilitating all system-specific applications. The CD storage platform enables the exemplary system to develop character-specific i.e., CD-specific programs that process only CCC and CC bracketed elements that prevent cross talk with matched elements disposed within shared operational platforms.

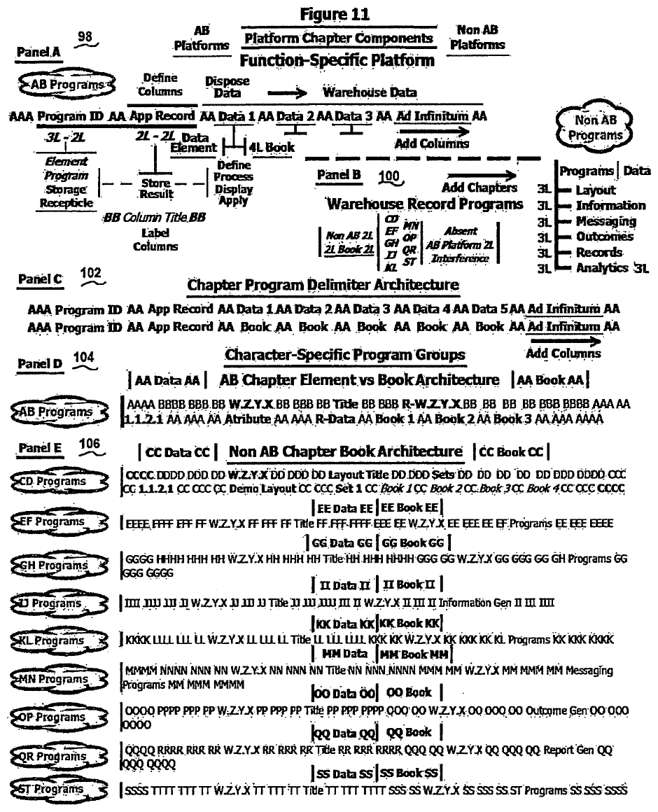

FIG. 11 is a schematic view that contrasts AAAA 4L AB-based operational platforms versus the AAA 3L chapter disposed 4L AB or 4L and 3L Non-AB-based platforms that make up the chapter-based ‘System of Records” with regard to each data-driven function. The platforms further include character-specific constructs to host character-specific programmed processes controlled by character specific 2L disposed data. In general AB operational platforms establish a multilevel Record Management System (RMS) that warehouse all data, programs and records respective of automating a respective data-driven function. Chapter-disposed programs include a respective function-specific ‘System of Record” made up of a plurality of Record Generators. Record Generators include but are not limited to the following: An Information generator that establishes a record of data, decisions and actions imbedded within ideally formatted informative text respective of the data and data-driven actions; Messaging generators respective of initiating real time guidance and governance of automated data-driven functions; automated outcome generators that monitor for and assimilate respective programmed outcomes that establish function-specific Records respective of each data-driven action supporting best practice analytics; and Record generators that collate historical data, decisions and actions with respective matched outcomes that fuel the Analytic programs respective of identifying Best Practices. Panel A 98 illustrates a 3L-AAA chapter disposed program comprised of at minimum a Program ID and unlimited warehousing of 2L bracketed data. The program further includes means to label columns beyond warehousing data respective of program specific applications. Panel B 100 exemplifies AAA 3L disposed non-AB programs as previously noted. Panel C 102 exemplifies distinguishing the first 2L disposed App Record from 2L disposed data as an Element or formatted within a delimiter-based Book Architecture. Panel D 104 illustrates that the 4L AB construct enables disposing dispose data as a data element or as a “System of Records” i.e., 4L book-construct. Panel E 106 exemplifies non-AB program constructs disposed in a cloud-based storage system that connects to a respective program Identifier.

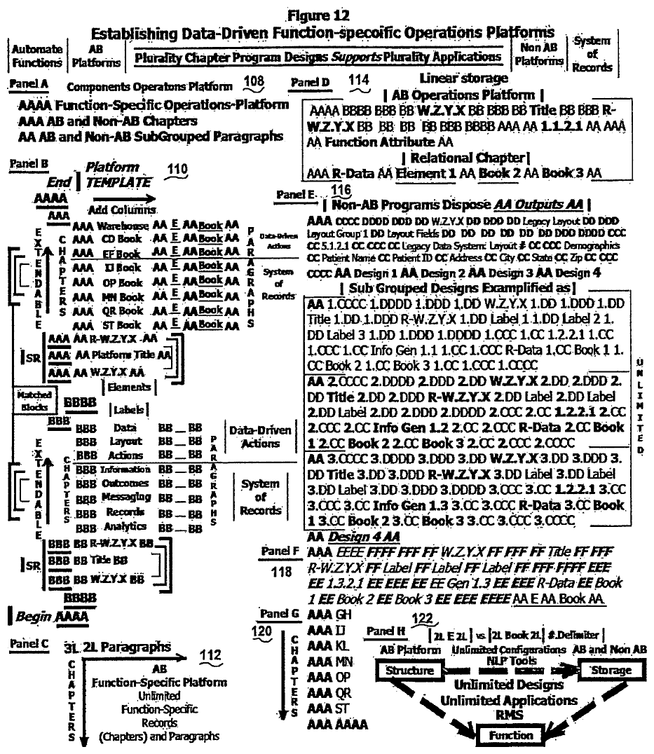

FIG. 12 exemplifies the operation of the exemplary system to establish an AB structured Data-driven, Function-specific, Operations Platform for each data-driven process with means for each Operations Platform to warehouse a plurality of data and chapter-disposed, data-driven, programmed processes comprised of both AB and Non-AB delimited structures. The structures collectively enable AB platform programs to automate respective data-driven functions. These features further include means to establish a common “System of Records” with regard to each operational platform represented in FIG. 11 that enable guidance, governance and best practice determinations individualized for each Non-AB programmed process and collectively with regard to the AB Operations-Platform completing a respective data-driven function. Panel A 108 represents the delimiter-disposed AB and non-AB based components of an AB Operations-Platform that conform with the previously described abstract book-architecture. This is further exemplified in Panel B110 that illustrates a delimiter-based skeletal-construct of an Operations Platform that segregates Data-driven actions and the “System of Records” that guide, govern and analyze for best practice. Panel C 112 illustrates the unlimited chapter-based and paragraph-based storage respective of both AB and non-AB operations platforms supporting automating any and all data-driven functions. Panels D-G exemplify linear storage of a variety of delimiter constructs that comprise an AB Operations Platform. Panel D 114 illustrates that Operations Platforms are morphed from the basic Storage-Record SR book-architecture of FIG. 5. Panel E 116 exemplifies a 3L, AAA-disposed Non-AB CD Layout program exemplified in FIG. 10 that initiates morphing a SR into an Operations-Platform and disposes four Layout designs AA disposed i.e., AA Design 1 AA Design 2 AA Design 3 AA Design 4 AA and constructed by means of the AAA disposed Layout-design Program. Each Design can be disposed by means of Design-specific identifiers means for delimiter-specific sub grouping exemplified as 1.CCCC, 2.CCCC and 3.CCCC. Panel F 118 illustrates an EF based construct prior to morphing into an Operations-Platform and demonstrates disposing either EE Elements EE or EE Book-constructs of Elements EE. Panel G 120 illustrates additional Non-AB pairings with each pairing offering unlimited numeric-based sub groupings as elucidated in Panel E 116. Panel H 122 illustrates the Ni P tools mediated primary structural designs of AB and Non-AB operations-platforms enabling disposing respective outputs as either unstructured or structured elements i.e. book architectures as well as subgrouping book architectures by means of affixing a numeric identifier to respective delimiters of a respective sub grouped construct. The collective enables NLP tools mediated unlimited configurations in structure enabling unlimited configurations in storage collectively enable unlimited operations-platform designs enabling unlimited applications managed by means of the Record Management System (RMS).

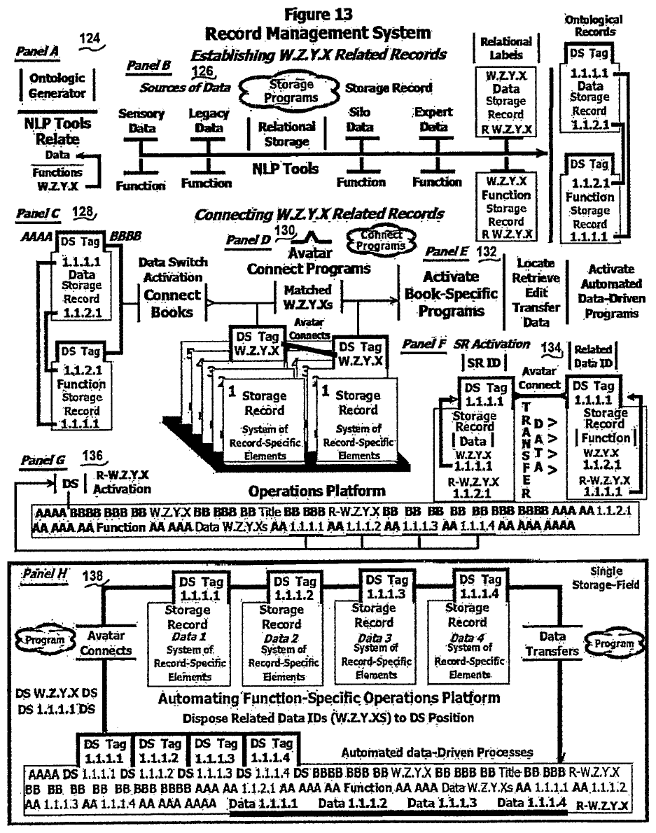

FIG. 13 illustrates by means of abstract structures the delimiter enabled components that enable the exemplary Record Management System (RMS) to connect Storage-Records of Data with Storage-Records of Function by disposed unique and related W.Z.Y.X identifiers. The system further provides capabilities for a W.Z.Y.X mediated “connection” activating “connect-programs” that transfer respective data from the “Storage-Record of Data” to the related “Storage-Record of Function” i.e., Operations Platform to activate respective data-driven functions. This capability provides for automating a function-specific “Operations Platform”. Panel A124 establishes that all system data is ontologically defined with regard to each of its assigned functions by means of Natural Language Programming (NLP) tools that disposes each unique W.Z.Y.X identifier of each respective function into each respective related Data-specific Storage-Record. It is further established that the collective of function-specific W.Z.Y.X identifiers are first chapter disposed and labeled as R-W.Z.Y.X. Panel B 126 illustrates a plurality of “Sources of Data” with each component data and function identified and labeled by means of NLP tools and ontologically related per Panel A 124 by means of “storage programs”. The storage programs construct the respective Storage Records of data and related functions exemplified as abstract paired related record structures at the end of Panel B 126. These abstract structures illustrate the W.Z.Y.X unique identifiers disposed as related W.Z.Y.X identifiers i.e., R-W.Z.Y.X in each respective related record.

Panel C 128 illustrates means for the system to connect related records by means of the “Data Switch” i.e., DS Tag illustrated as an abstract record structure. Panel D 130 illustrates connecting related records by means of matched W.Z.Y.X identifiers disposed as DS Tags i.e., bracketed between the first odd character 4L delimiter and the first even character 4L delimiter i.e., between the borders of the Storage-Record and the Table of Contents of the respective SR (Panel C). The Data Switch represented in Panel E 132 enables the system to activate an SR by means of disposing a W.Z.Y.X identifier or inactivate by means of removing a W.Z.Y.X. “Avatar Connect Programs” are generic location programs respective of matching Data Switch disposed W.Z.Y.X identifiers. This system further includes means for an “Avatar Connect” to activate programs assigned to a connected Storage Record that complete data transfers and activation of assigned programs collectively automating data driven programs. Linear storage of all Storage Records within a single storage-field or equivalent without data-field partitioning enables a single generic “Avatar Connect Program” to be activated by means of disposing a respective W.Z.Y.X identifier. The system further includes means for activation to enable Avatar-Connects with any and all Storage-Records activated for “Connect” by means of a matching Data Switch disposed W.Z.Y.X identifier. Panel F 134 exemplifies Data-Switch activation by means of disposing the 1.1.1.1 W.Z.Y.X identifier further including the “Avatar Connect” to the matched DS Tag 1.1.1.1 that enables the transfer of data. Panel G 136 exemplifies sequenced storage of a Storage Record that is activated by means of disposing copies of the related W.Z.Y.Xs from the R-W.Z.Y.X chapter to the DS Tag position. Panel H 138 illustrates each of the four activated Data-specific Storage-Records to “Avatar Connect” with the respective DS Tag data-specific W.Z.Y.X identifier i.e., (DS W.Z.Y.X DS) exemplified as (DS 1.1.1.1 DS) that enables transferring their respective data to the function-specific Operations Platform to complete the respective disposed data-driven programmed functions.

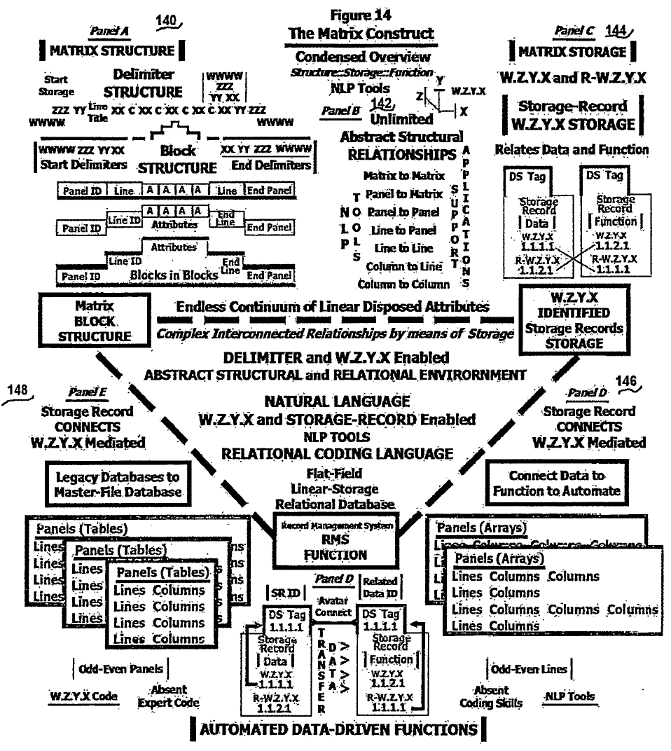

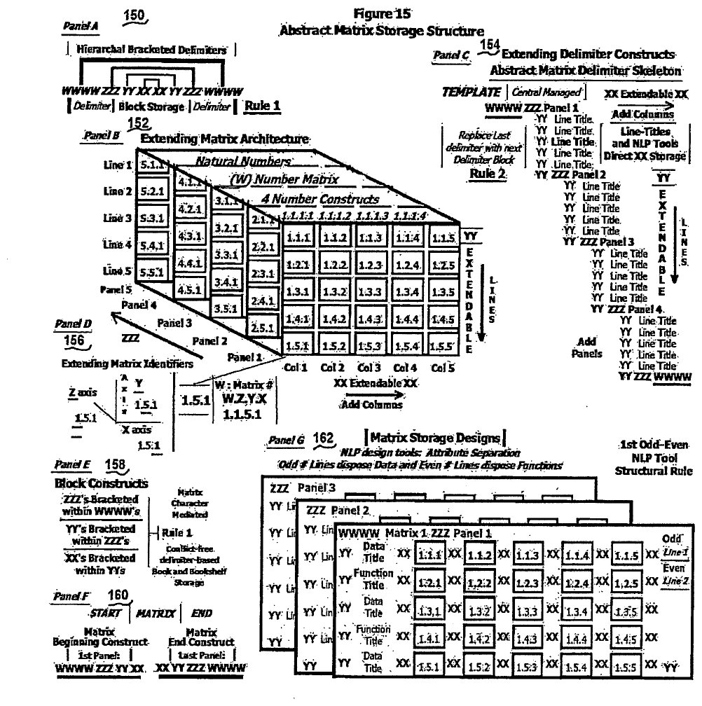

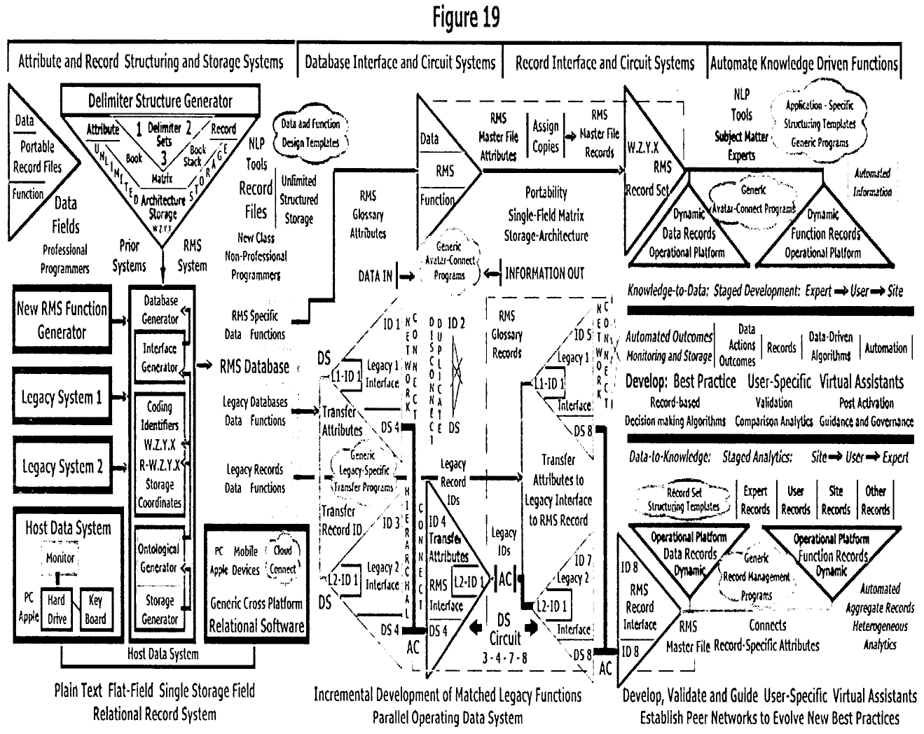

FIG. 14 presents a simplified condensed overview of “The Matrix-Construct” illustrating the major components respective of the system's design-trilogy comprised of relating structure, storage and function. Each abstract matrix-construct is established by repetitive sequencing of four matrix-specific alphanumeric delimiters that include XX, YY, ZZZ and WWWW respectively. This further includes means for NLP tools to utilize block-storage to construct abstract configurations respective of columns, lines, panels and tables that collectively establish a respective abstract Matrix-structure exemplified in Panel A 140. Panel B 142 illustrates how the system uniquely identifies each respective matrix-coordinate of a four component, natural number nodal-construct i.e., labeled as W.Z.Y.X. This value identifies each respective Z.Y.X unique nodal-intercept identifying each disposed Storage-Record of each unique matrix-construct uniquely identified as (W). NLP tool enabled Object-based columns, lines and panel abstractions enable one or a plurality of object-based, multilevel, shared relationships orchestrated within each respective attribute-specific Storage-Record by means of disposed W.Z.Y.X identifiers collectively supporting portable, complex, interactive, attribute-specific functions. Panel C 144 exemplifies disposing the W.Z.Y.X identifiers of related attributes within each respective attribute-specific Storage-Record by using NLP tools to establish a Natural Language “Relational Coding Language” without expert code or expert coding skills. This enables connecting odd-line disposed storage-records of data to even-line disposed storage-records of function (FIG. 13) to automate data-driven functions Panel D 146. Panel E 148 illustrates means to connect Legacy Data Systems to the data system by means of related Legacy specific and system specific mirrored panels of matched Storage-records. In this arrangement one panel is respective of Legacy data and the matched panel is respective of a matched system representation of the Legacy data. Shared W.Z.Y.X identifiers nodal-connect Legacy and system storage-records to share data across previously unconnected Legacy data systems. The system further includes means to establish a Master-File System database by means of relating a plurality of Legacy data-specific storage-records to a single Storage-Record comprising the Master-File (Panel E). FIGS. 15-19 will further describe each of the components comprising “The Matrix-Construct”.

FIG. 15 describes in the key structural components and respective functional attributes of the exemplary system's delimiter abstract-matrix storage design. This design enables unlimited storage and illustrates the W.Z.Y.X matrix-specific identifiers to identify, relate and connect respective disposed delimiter constructs comprising abstract books and bookshelf designs. Panel A 150 illustrates the Rule 1 mediated delimiter “Block-Storage” i.e., block in block bracketed storage design of the matrix-specific delimiters i.e., WWWW, ZZZ, YY and XX. Panel B 152 illustrates the delimiter-based abstract matrix design that enables extending the matrix storage-architecture by means of adding ZZZ-based Panels of YY-based Lines and XX-based columns each extendable by means of replacing the last respective delimiter with the next respective delimiter-block per Rule 2 (FIG. 5, Panel D). Panel C 154 illustrates a respective delimiter matrix-design Template that establishes a delimiter skeleton that includes programmed Line-Titles to direct disposing type-specific groups of Book and Bookshelf record-constructs by means of the system's Natural Language Programming (NLP) tools. Templates and NLP tools can be centrally designed to ideally guide and support decentralized storage-record designs and mediated functions. Panel D 156 illustrates the Z.Y.X numeric intercept afforded by the matrix architecture, that combined with the numeric identifier of the matrix, establishes the W.Z.Y.X unique identifier exemplified by (1.1.1.5) of each disposed book or bookshelf record-construct thereby uniquely identifying each record-construct. 1.1.1.5 identifies first matrix, first panel, first line and fifth column matrix-specific storage site.

Panel E 158 further describes Rule 1 non-hierarchal, character-based, delimiter bracketing of XX-Blocks within YY-blocks within ZZZ-blocks within WWWW-blocks. W, X, Y and Z character constructs eliminate delimiter-based storage conflicts of Book and Bookshelf constructs and the respective programs assigned to each character-specific “Operations Platforms” (FIG. 10 Panel D). Block-storage per delimiter construct Rules 1 and 2 enable identifying the beginning and the end of each respective Matrix-storage group by means of [WWWW ZZZ YY XX] and [XX YY ZZZ WWWW], respectively exemplified in Panel F 160. Panel G 162 illustrates a matrix storage design and the first NLP tool mediated structural Rule to facilitate disposing data within odd numbered line YY constructs and disposing respective related data driven process within the next sequenced even numbered YY line construct. W.Z.Y.X identifiers relate data respective of a defined function by means of Y=1 and Y=2, respectively i.e., W.Z.1.X and W.Z.2.X, respectively exemplifying how the system processes of structure and storage support NLP tools to design, relate and connect data to respective data-driven functions. Enterprise Line-Title Templates guide regional designs of data and functions.

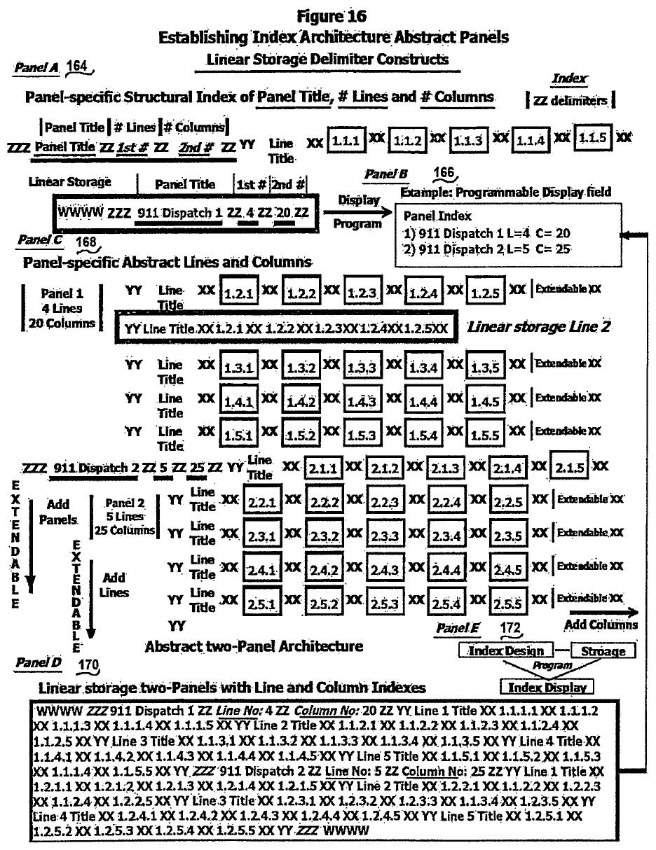

FIG. 16 establishes the Panel-specific delimiter designed Index of lines and columns comprising a respective panel utilizing the ZZ delimiter to dispose the respective line and column numbers of each panel construct as illustrated in Panel A 164. Index numbers are utilized to program a “programmable display field” to list panel titles and respective line numbers and column numbers as exemplified in Panel B 166. The abstract structures of panels one and two are illustrated in Panel C 168 that includes linear disposition of panel 1, line 2 delimiters and example Z.Y.X nodal identifiers. Both panel examples contain five lines and 20 columns with means to extend the number of disposed panels and respective lines and column specific arrays included in each panel. Panel D 170 exemplifies linear storage of delimiters and respective elements comprising the abstract panel structures of Panel C 168 inclusive of the panel-specific Index of each panel fueling the programmed image in Panel B 166. Panel E 172 exemplifies the relationships between index structure, storage and a display-specific function.

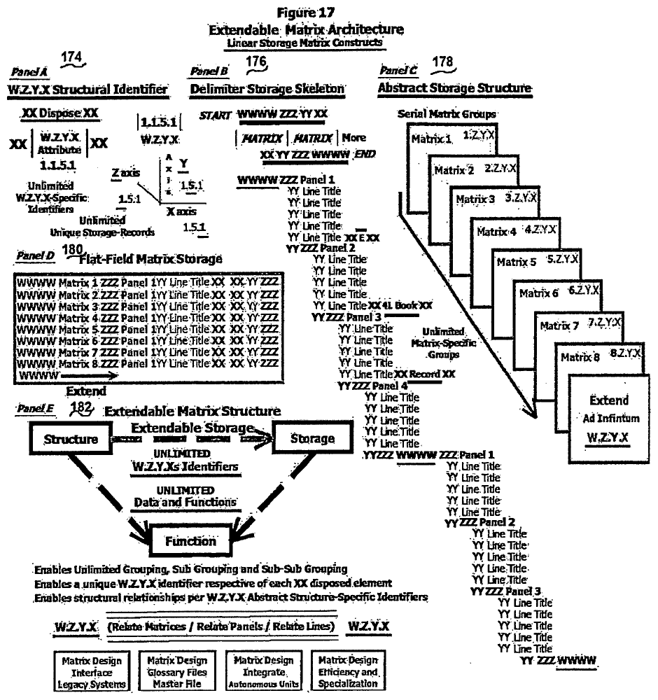

FIG. 17 illustrates the linear extendibility of matrix delimiter constructs to dispose major attribute groups separately and together within the same storage-field or equivalent. The matrix architecture enables unlimited W.Z.Y.X unique identifiers of each matrix construct to uniquely identify and dispose unlimited numbers of attributes respective of each matrix construct as illustrated in Panel A 174. The extendable matrix skeleton of delimiters is exemplified in Panel B 176 noting that every linear-disposed data system has a starting and ending delimiter sequence bracketing innumerable matrix architectures i.e., block in block design. Panel B 176 further illustrates XX delimiter bracketing of abstract columns of (E) elements, (4L Book) Storage-Record constructs and/or (Records) i.e., a plurality of shared related Storage-Record constructs disposed within 5L, 6L, and greater delimiters (FIG. 3, Panel B). Serial disposed abstract matrix architectures are illustrated in Panel C 178 exemplifying sequencing the natural number (W) identifier respective of sequenced matrix storage with unlimited matrix groupings. Panel D 180 represents linear storage of matrix delimiters respective of the abstract matrix architecture. Panel E 182 illustrates the triad of structure, storage and function with matrix structure enabling unlimited matrix-enabled unlimited W.Z.Y.Xs identified attribute storage supporting unlimited attributes of function including means for unlimited abstract structural relationships of disposed and W.Z.Y.X labeled delimiter constructs comprising lines, panels and matrixes. Each abstract structural relationship is identifiable by means of the respective matched or related matrix, panel or line identifier i.e., (W), (Z) and (Y) of the W.Z.Y.X identifier construct. Collectively these relationships enable the data system to connect data and respective data-driven functions. The construct further includes means to interface the data systems with Legacy data systems including means to collate the plurality of Legacy attribute-glossaries into a single Invention Master-File. The W.Z.Y.X mediated relationships establish a Nodal-Network of related W.Z.Y.X identifiers to nodal connect Data-Switch (DS) disposed related W.Z.Y.X identifiers enabling the system to route attributes across previously unconnected Legacy data systems and to connect attributes of data with related attributes of function enabling data-driven automation of respective data-driven functions.

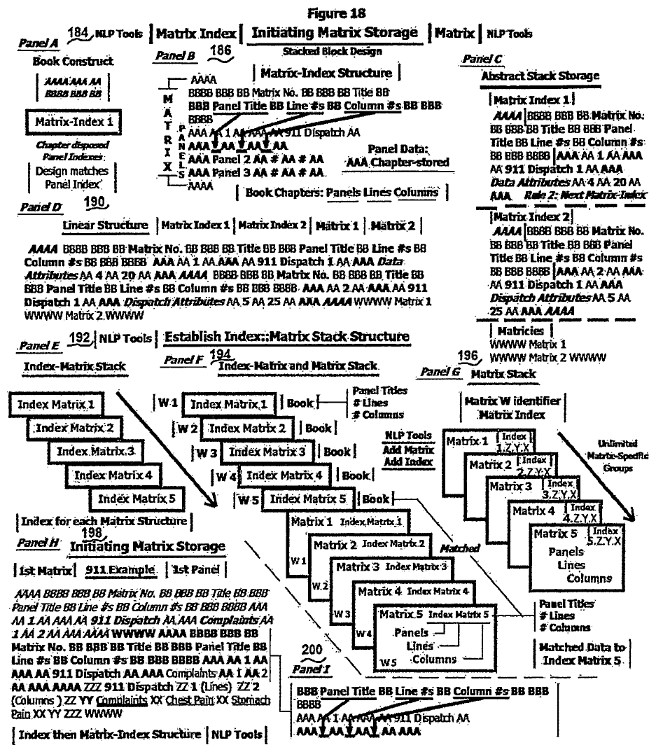

FIG. 18 illustrates how the exemplary system builds matrix storage by means of a stacked-block design. The storage is constructed by Natural Language Programming tools that begins with a sequenced stack of Matrix-Indexes followed by a sequence matched stack of matrix-groups. Panel A 184 illustrates that each Matrix-Index is formatted by means of an AB book construct with respective Panel-Indexes chapter disposed. Panel B 186 exemplifies Matrix-Index structure with A character delimiters disposed data defined by B character delimiter disposed “Table of Contents. Data includes the matrix-Number i.e., (W) and the matrix-Title with each panel-title chapter-disposed and 2L delimiters bracketing the panel-specific Line-number and Column-numbers.

Panel C 188 illustrates the abstract stack-design Matrix-Indexes followed by Matrix constructs. Both Index and Matrix stacks are extendable by means of Rule 2 i.e., replace the last AAAA or WWWW respectively with the next AAAA bracketed Matrix-Index and the next WWWW bracketed Matrix group. Panel D 190 exemplifies linear-storage of the abstract stacks in Panel C. Panels E 192, F 194 and G 196 exemplifies extendable abstract stack designs for unlimited matched extensions of the Matrix-Index and Matrix structures through the use of NLP tools. Panel F 194 illustrates disposing a matched Matrix-Index within each Matrix-construct. Panel H 198 exemplifies Initiating Matrix storage architecture by means of beginning to build a sample program titled 911 Dispatch. An AAAA bracketed Matrix Index i.e., [AAAA BBBB BBB BB Matrix No. BB BBB BB Title BB BBB Panel Title BB Line Ifs BB Column BB BBB BBBB AAA AA 1 AA AAA AA 911 Dispatch AA AAA Complaints AA 1 AA 2 AA AAA AAAA] is followed by a WWWW bracketed “911 Dispatch” titled Matrix. The first WWWW matrix delimiter is followed by a matched copy of the Matrix-Index i.e., [WWWW AAAA BBBB BBB BB Matrix No. BB BBB BB Title BB BBB Panel Title 1313 Line its BB Column #s BB BBB BBBB AAA AA 1 AA AAA AA 911 Dispatch AA AAA Complaints AA 1 AA 2 AA AAA AAAA]. The last AAAA is followed by the first ZZZ bracketed matrix-panel i.e., [ZZZ 911 Dispatch ZZ 1 (Lines) ZZ 2 (Columns) ZZ YY Complaints XX Chest Pain XX Stomach Pain XX ZZZ] that includes the respective ZZZ Panel Index i.e., [ZZZ 911 Dispatch ZZ 1 (Lines) ZZ 2 (Columns) ZZ] followed by the Line attributes i.e., [YY Complaints XX Chest Pain XX Stomach Pain XX]. As previously noted the [XX YY ZZZ WWWW] delimiter construct marks the end of a respective Matrix.

Of course it should be understood that the above described system features are exemplary, and other systems may be devised which employ the inventive features and approaches that have been discussed herein. Further it should be understood that while the exemplary system has been described in connection with improving operations in healthcare related organizations, the approaches described may also be implemented within other organizational structures of other types of organizations and enterprises.

Thus the exemplary embodiments achieve improved operation, eliminate difficulties encountered in the use of prior devices and systems, and attain the useful results described herein.

In the foregoing description certain terms have been used for brevity, clarity and understanding. However, no unnecessary limitations are to be implied therefrom because such terms are used for descriptive purposes and are intended to be broadly construed. Moreover, the descriptions and illustrations herein are by way of examples and the inventive features are not limited to the exact features shown and described.

Having described the features, discoveries, and principles of the exemplary embodiments, the manner in which they are constructed and operated, and the advantages and useful results attained, the new and useful structures, devices, elements, arrangements, parts, combinations, systems, equipment, operations, methods, processes and relationships are set forth in the appended claims.

We claim:

1. A computer operable records management system comprising:
a computer operable data storage memory medium operably linked to a computer processor;
a plurality of records documenting transactions performed in a business operation;
a record structure hierarchy containing the plurality of records and comprising a plurality of levels stored on the computer operable memory;
an algorithm operable on the computer processor which retrieves the plurality of records, compiles recurring processes, tabulates and organizes recurring processes, recognizes patterns occurring in the recurring processes, and generates best business practices documentation based on the patterns occurring in the recurring processes.

2. The system of claim 1 wherein the record structure hierarchy further comprises a plurality levels in a tree, branch and leaf configuration.

3. The system of claim 2 wherein the plurality of levels further comprise a first level comprised of alphabetic characters “a” through “t” and a second level comprised of characters “u” through “z” and a third level comprised of unmatched characters.

4. The system of claim 3 further comprising each string comprised of a single character with one set that features the character sequence (W Z Y X) and all sets features Y and X as third and fourth hierarchal characters with each storage language to feature upper and lower case character combinations with at least one set of all upper case characters respective of each character.

5. The system of claim 2 wherein the plurality of levels further comprise a group, a sub group and a sub-sub group of function-specific levels comprised of alpha-numeric characters.

6. The system of claim 2 wherein the plurality of levels further comprise character strings which establish three non-correlative string lengths with a first level string and a third level string comprising string lengths of four, three and two characters, and a second level string which contains more than four characters.

7. The system of claim 2, wherein the plurality of levels further comprise strings correlative left to right highest to lowest and lowest to highest delimiter which establish a storage block respective of each sequence of matched delimiters with lower blocks bracketed within higher blocks three string constructs with two tiers of metadata and LSL-3 featuring four string constructs with three tiers.

8. The system of claim 2, wherein an administrative record which records an administrative functions is denoted by the character AB.

9. The system of claim 2 wherein a non-correlative data storage modules enables the data structure to define data as generic such that data is separated as identifier that assign ownership and a separate identifier identifies data enabling data to be accessed with out regard to ownership.

10. The system of claim 9, wherein the odd numbered delimiter comprises templates.

11. The system of claim 9, wherein the even numbered row comprises matched labels.

12. A computer operable medical records management system comprising:
a computer operable data storage memory medium operably linked to a computer processor;
a plurality of medical records documenting transactions performed in a business operation;
a record structure hierarchy containing the plurality of medical records and comprising a plurality of levels stored on the computer operable memory;
an algorithm operable on the computer processor which retrieves the plurality of medical records, compiles recurring processes, tabulates and organizes recurring processes, recognizes patterns occurring in the recurring processes, and generates best business practices documentation based on the patterns occurring in the recurring processes.

13. The system of claim 12 wherein the medical record structure hierarchy further comprises a plurality of levels further comprise an admission station, a diagnosis station and a patient room station.

14. The system of claim 2 wherein the plurality of levels feature a first level comprised of a set of alphabetic characters and a second level comprised of different alphabetic characters and a third level comprised of unmatched characters.

15. The system of claim 3 further comprising each string comprised of a single character with one set that features the character sequence and all sets features as third and fourth hierarchal characters with each storage language to feature upper and lower case character combinations with at least one set of all upper case characters respective of each character.

16. The system of claim 2 wherein the plurality of levels further comprise a group, a sub group and a sub-sub group of function-specific levels comprised of alpha numeric characters.

17. The system of claim 2 wherein the plurality of levels further comprise character strings which establish three non-correlative string lengths with a first level string and a third level string comprising string lengths of four, three and two characters, and a second level string which contains more than four characters.

18. A flat-field, natural language, Record Management System (RMS) that digitalizes an organization's current structures, facility knowledge and systems of function with records purposed to idealize each system's best practice function and records purposed to automate, guide and govern validated data-driven best practice functions and evolve new best practice structures and systems of function, the system comprising:
A computer data system comprised of a memory, a monitor, a readable storage medium and at least one user interface that hosts a Generic Relational Database (GRD) in operative connection with a single designated storage field or equivalent (DSF) that enables programming the RMS and Natural Language Programming (NLP) tools;
Three non-correlative Declarative Record Structures (DRS) with correlative extendable Hierarchal Indexed tables (HIT) that enable subject matter experts (SME) absent expert programming skills to code unlimited mutable metadata, data and processes with NLP tools that comprise unlimited structure-specific programmed functions managed by the RMS with all DRSs structure-stored in the DSF either separately or nested within the same DSR or another DSR with an index network managed by the RMS that connects all DRSs and respective HITs that enable the RMS to guide, govern, validate and automate all components of all functions of the Linear Record Data System (RDS);
Three Declarative Record Structures (DRS) with rule based character and string length assignments that establish three non-correlative, flat-field, linear delimiter lexicon storage languages (LSLs) that structure-store data and tiered metadata in correlative declarative delimiter structures that enable structure-in-structure non hierarchal nested storage with block extensions that establish correlative hierarchal Indexed tables (HIT) of metadata and data that enable correlated data exchanges of unlimited data and metadata stored separately, together and indexed within lexicon record HIT constructs stored separately, together and indexed within a DSF;
Non-alphanumeric lexicon storage languages (NASL) index programmed elements that can declare a function and a method inside a structure that define and control site-specific and function-specific metadata and data in distributed record structures;
NLP tools in operative connection with the RMS and the DSF ontologically store LSL data and metadata in an extendable contiguous single digital space storage nexus (DSSN) with subject matter expert (SME) defined, function-specific, cluster stored, ontological tree, branch and leaf data and metadata that comprise LSL record constructs uniquely identified and ontologically defined with storage of unique index storage nodes (ISNs) together with stored hierarchal labels that support distributed applications engineered with RMS distributed ISNs that network LSL data and metadata with each ISN network structure-activated by the RMS to establish structure-specific and function-specific index circuitry that guide, govern, validate and automate HIT aggregate record functions (ARFs) that comprise SME designed best practice knowledge work (BPKW) bottom-up, incrementally programmed and DSSN stored with NLP tools absent expert programming skills with site-specific SMEs that program site-specific NASLs with NLP tools that idealize site-specific BPKW with automated informative reports (AIRs) and outcome record analytics (ORAs) ever evolving new site-specific BPKW and heterogeneous best practice analytics (BPAs) ever evolving new BPKW respective of each SME and the DSSN record stored in the DSF; and
Bottom-up staged modular development establishes a DSSN monolith record comprised of clustered modular best practice ARFs that enable non DSF (NDSF) ARFs storage with unlimited group, sub group and sub-sub group partitioning that enable DSF language one record constructs to aggregate unlimited function-specific aggregate sequenced ARFs to manage new BPKW within the DSF and external to the DSF with unlimited cloned deployments of DSW DSSN modular ARFs guided, governed and updated via the common reference DSW DSSN that ever evolves distributed non DSF site-specific BPKW with non DSF distributed data fueling heterogeneous DSF DSSN aggregate data that ever evolves new DSF DSSN BPKW.

19. The system of claim 18 further comprising three groups of lexicographic hierarchal delimiter sets with rule based alpha character and string length assignments that establish three non-correlative, flat-field, linear LSLs according to claim 1 feature discordant characters with LSL-1 and LSL-2 that feature alpha characters “a” through “t” and LSL-3 characters “u” through “z” with LSL-1 and LSL-2 delimiter sets comprised of character matched strings with LSL-3 delimiter strings comprised of unmatched characters with each string comprised of a single character with one set that features the character sequence (W Z Y X) and all sets feature y and x as third and fourth hierarchal characters with each storage language to feature upper and lower case character combinations with at least one set of all upper case characters respective of each character.

20. The system of claim 18 further comprising numeric substitutes of alpha characters that comprise the bracketing delimiters of each LSL construct according to claim three establish function-specific classes respective of each LSL that can be grouped sub grouped and sub-sub grouped and include matched substitutions of the middle characters comprising the end brackets of all three LSL constructs with no change to the alpha delimiters that comprise the lower tiered delimiter blocks and matched numeric substitutions of the end character comprising the end brackets of all three LSL constructs with no change to the middle alpha characters and no change to the alpha delimiters that comprise the lower tiered delimiter blocks.
